(19) United States
(12) Patent Application Publication
(10) Pub. No.: US 2017/0035823 A1
(43) Pub. Date: Feb. 9, 2017

(54) METHOD FOR TREATING OR SUPPRESSING DEGENERATIVE BRAIN DISEASES BY CHINESE HERBAL MEDICINE COMPOSITION

(71) Applicant: Brion Research Institute of Taiwan, New Taipei City (TW)

(72) Inventors: Shuenn-Jyi SHEU, New Taipei City (TW); Chung-Hsin WU, Taipei City (TW)

(21) Appl. No.: 15/044,359

(22) Filed: Feb. 16, 2016

(30) Foreign Application Priority Data
Aug. 5, 2015 (TW) 104125445

(51) Int. Cl. A61K 36/258; A61K 36/481; A61K 9/00; A61K 36/28; A61K 36/64; A61K 36/8988; A61K 36/232; A61K 36/638

(52) U.S. Cl. CPC A61K 36/258; A61K 36/232; A61K 36/481; A61K 36/638; A61K 36/28; A61K 36/64; A61K 36/8988; A61K 9/0053; A61K 2236/333; A61K 2236/53; A61K 2236/331

(57) ABSTRACT

The present invention discloses a method for treating or suppressing degenerative brain diseases, comprising a step of administrating a Chinese herbal medicine composition to a subject in need, wherein the Chinese herbal medicine composition comprises: a first herbal formula comprising 2-40 parts by weight of Panax ginseng and 0.6-28 parts by weight of Angelica sinensis; and a second herbal formula comprising 0.6-28 parts by weight of Angelica sinensis, 2-40 parts by weight of Astragalus membranaceus, and 20-96 parts by weight of a mixture of Ligustri fructus, Eclipta prostrata, and Rehmannia glutinosa.

### CROSS REFERENCE TO RELATED APPLICATION

This application claims the benefits of the Taiwan Patent Application Serial Number 104125445, filed on Aug. 5, 2015, the subject matter of which is incorporated herein by reference.

### BACKGROUND OF THE INVENTION

1. Field of the Invention

The present invention relates a method for treating or suppressing degenerative brain diseases, such as Huntington's disease, cerebrovascular dementia, Alzheimer's disease, or Parkinson's disease, using a Chinese herbal medicine composition.

2. Description of Related Art

Dementia is an important issue of the society. Due to factors such as aging population and changing environment, dementia has increased over the years in Taiwan. According to current researches, there are four subtypes of dementia, which are Alzheimer's disease (AD), vascular dementia (VaD), Dementia with Lewy Bodies (DLB), frontotemporal lobe degeneration (FTD), listed by the frequency of occurrence. As reported by the World Health Organization in 2010, Alzheimer's disease and its related diseases accounted for almost 90% of dementia. This indicates that the majority of the dementia patients are with Alzheimer's disease. However, the exact cause of the disease is still unclear. In general, it is believed that cerebrovascular disease is an important cause of dementia. When cerebral blood circulation is insufficient and when nutrient supply (such as oxygen, glucose) is in shortage, hippocampal neuronal cells will be damaged. The risk of developing dementia is subsequently increased.

Another neurodegenerative disease of the brain is Huntington's disease (HD). Huntington's disease is caused by the abnormality of the fourth chromosomal pair. Specifically, the excessive expansion of CAG repeats causes brain degeneration. Huntington's disease is characterized by uncontrolled limbs with decreasing intelligence and eventually death caused by reasons such as difficulties in swallowing and breathing. Up until now, pharmaceuticals can only slow down but cannot stop brain degeneration. There is still no effective treatment to stop or to recover from Huntington's disease.

Parkinson's disease (PD) is a chronic and progressive movement disorder, meaning that symptoms continue and worsen over time. The cause is unknown, and although there is presently no cure, there are treatment options such as medication and surgery to manage its symptoms. PD primarily affects neurons in an area of the brain called the substantia nigra. Some of these dying neurons produce dopamine, a chemical that sends messages to the part of the brain that controls movement and coordination. As PD progresses, the amount of dopamine produced in the brain decreases, leaving a person unable to control movement normally. Since the brain is a very delicate and sophisticated organ and since the causes of degenerative brain diseases are still unclear; thus, related researches are still ongoing. However, the therapeutic effects of the existing pharmaceuticals for the suppression or treatment of degenerative brain diseases are very limited. The existing pharmaceuticals can only slow down the progression of degenerative brain diseases but cannot cure degenerative brain disease. Hence, efforts still need to be devoted to the development of other pharmaceuticals or methods that can effectively suppress and even to treat degenerative brain diseases.

### SUMMARY OF THE INVENTION

Although the current treatment or suppression of degenerative brain diseases mainly uses pharmaceuticals; however, Chinese herbal medicine can also be a new choice for the treatment or suppression of degenerative brain diseases. One of the reasons is that Chinese herbal medicine has good therapeutic effects as well as fewer side effects.

To achieve the object described above, the present invention provides a method for treating or suppressing degenerative brain diseases using a Chinese herbal medicine composition. The Chinese herbal medicine composition, which can protect nerve cells, has a great potential in treating or suppressing degenerative brain disease.

In the present invention, “treatment” means when the management of abnormal body conditions (such as symptoms, abnormalities, or diseases) of human beings or animals involves treatment or therapy to achieve certain desired efficacy. For example, the suppression of abnormal body conditions includes slowing down disease progression, stopping disease progression, improving abnormal body conditions, mitigating abnormal body conditions, and eliminating abnormal body conditions. In other words, treatment can mean the elimination of one or more symptoms of a disorder or the complete eradication of a disorder.

“Prevention” means when the management of abnormal body conditions (such as symptoms, abnormalities, or diseases) of human beings or animals involves prevention or inhibition to achieve certain desired preventive effect. For example, prevention means to prevent onsets of diseases or to be uninfected by diseases. Prevention includes completely and overall stopping all symptoms of a disorder in an unscheduled way, delaying the onsets of one or more symptoms of a disease, or causing the onsets of diseases to occur less easily in an individual.

In the present invention, a method for treating or suppressing degenerative brain diseases is provided, which comprises a step of administrating a Chinese herbal medicine composition to a subject in need, wherein the Chinese herbal medicine composition comprises: a first herbal formula comprising 2-40 parts by weight of Panax ginseng and 0.6-28 parts by weight of Angelica sinensis; and a second herbal formula comprising 0.6-28 parts by weight of Angelica sinensis, 2-40 parts by weight of Astragalus membranaceus, and 20-96 parts by weight of a mixture of Ligustri fructus, Eclipta prostrata, and Rehmannia glutinosa.

More specifically, the Chinese herbal medicine composition of the present invention comprises the first herbal formula comprising 20-40 parts by weight of Panax ginseng and 0.8-20 parts by weight of Angelica sinensis and the second herbal formula comprising 1-24 parts by weight of Angelica sinensis, 2-40 parts by weight of Astragalus membranaceus, and 20-96 parts by weight of the mixture of Ligustri fructus, Eclipta prostrata, and Rehmannia glutinosa. A weight ratio of Ligustri fructus, Eclipta prostrata, and Rehmannia glutinosa in the mixture is 0.8-1.2:0.8-1.2:0.8-1.2, and more preferably, the weight ratio of Ligustri fructus, Eclipta prostrata, and Rehmannia glutinosa in the mixture is 1:1:1.

The Chinese herbal medicine composition of the present invention may further comprise an excipient, which may be any known excipient, such as an excipient selected from a group consisting of starch, corn starch, lactose, microcrystalline cellulose, carboxymethylcellulose, sucrose, mannitol, talcum powder, and magnesium stearate. The excipient may also be other known excipients used in pharmaceuticals or food. When the Chinese herbal medicine composition of the present invention comprises an excipient, the Chinese herbal medicine composition may be in different dosage forms depending on different needs, such as granules, capsules, pills, tablets, to facilitate feeding and to even enhance efficacy.

The Chinese herbal medicine composition of the present invention may further comprise a sweetening agent, a flavoring agent, or a coloring agent depending on different needs. However, the present invention is not limited thereto.

Gastrodiae Rhizoma can calm the brain. Gastrodiae Rhizoma also has analgesic effect, antispasmodic effect, heart and liver protection function, hypotensive effect, blood circulation improvement function, anti-inflammatory effect, immunity enhancement function, anti-aging effect, hypoxia tolerance increasing effect, skin temperature increasing effect, and intestinal excitatory effect. Therefore, another example of the Chinese herbal medicine composition of the present invention may further comprise 1-10 parts by weight of Gastrodiae Rhizoma.

In the method for treating or suppressing degenerative brain diseases provided by the present invention, a route of administration of the Chinese herbal medicine composition to a subject in need may be oral administration or injection administration. Although the route of administration of the Chinese herbal medicine composition of the present invention is not particularly limited; however, oral administration is preferred.

In the present invention, the degenerative brain diseases that can be treated or suppressed by the method provided are at least one of the Huntington's disease, cerebrovascular dementia, Alzheimer's disease, Lewy body dementia, frontotemporal dementia, Parkinson's disease, or other known degenerative brain diseases. More specifically, the degenerative brain disease that can be treated or suppressed by the method provided by the present invention may be Huntington's disease or Alzheimer's disease.

In the present invention, to ensure the Chinese herbal medicine composition can have better efficacy, the Chinese herbal medicine composition is prepared by the following method, comprising: (A) providing a first herbal formula and a second herbal formula, wherein the first herbal formula comprises 20-40 parts by weight of Panax ginseng and 0.8-20 parts by weight of Angelica sinensis and the second herbal formula comprises 0.6-28 parts by weight of Angelica sinensis, 2-40 parts by weight of Astragalus membranaceus, and 20-96 parts by weight of a mixture of Ligustri fructus, Eclipta prostrata, and Rehmannia glutinosa; (B) extracting and filtering the first herbal formula and the second herbal formula to obtain a first extract and a second extract, respectively; (C) filtering the second extract to obtain a third extract; and (D) mixing the first extract and the third extract.

In the above-mentioned method of the present invention, in step (B), the first herbal formula may be extracted using alcohol. A concentration of the alcohol may be 30-80 vol %, but preferably, 30-70 vol %. The alcohol may be ethanol. The second herbal formula may be extracted using water, such as decocting with boiling water.

In the aforementioned method of the present invention, in step (B), any known methods may be used to filter and separate macromolecules of herbs, such as the method of extraction by water and precipitation by alcohol, filtration by centrifugation, filtration by ceramic membranes, and methods using enzymes. However, the present invention is not limited thereto as long as the macromolecules of herbs can be filtered and separated effectively.

In the aforesaid method of the present invention, the third extract may be obtained by filtration using ceramic membranes.

In the present invention, the Chinese herbal medicine composition used to treat or suppress degenerative brain diseases comprises many Chinese herbal medicines in specific composition ratios. The Chinese herbal medicine composition can promote microcirculation effectively, and increase blood flow in the brain, causing the supply of nutrients to the brain to be more efficient. The Chinese herbal medicine composition also has anti-inflammatory effect and free radical elimination function, reducing oxidative stress in the brain. Accordingly, the Chinese herbal medicine composition can protect nerve cells and can thereby treat or suppress degenerative brain diseases.

Other objects, advantages, and novel features of the present invention will become more apparent from the following detailed description when taken in conjunction with the accompanying drawings.

### BRIEF DESCRIPTION OF THE DRAWINGS

FIGS. 1A-1E show the effective delaying or prevention of nerve cell atrophy caused by excitotoxic killing of SH-SY5Y neuroblastoma cells induced by glutamate by a Chinese herbal medicine composition of the present invention;

FIGS. 2A-2C show the survival, body weight, and motor function of the R6/2 transgenic Huntington's Disease (HD) mice treated by a Chinese herbal medicine composition of the present invention;

FIGS. 3A-3C show the enhanced microcirculation of the R6/2 transgenic HD mice treated by a Chinese herbal medicine composition of the present invention;

FIG. 4A shows the brain structure of 3-month HD mice studied by magnetic resonance image (MRI);

FIGS. 4B-4C show the alleviated brain atrophy of the R6/2 transgenic HD mice treated by a Chinese herbal medicine composition of the present invention;

FIGS. 5A-5B show the downregulated huntingtin aggregation in the brain tissue of the R6/2 transgenic HD mice treated by Chinese herbal medicine composition of the present invention;

FIGS. 6A-6B show the upregulated Brain-Derived Neurotrophic Factor (BDNF) levels in the brain tissue of the R6/2 transgenic HD mice treated by a Chinese herbal medicine composition of the present invention;

FIGS. 7A-7B show the upregulated Vascular Endothelial Growth Factor (VEGF) levels in the brain tissue of the R6/2 transgenic HD mice treated by a Chinese herbal medicine composition of the present invention;

FIGS. 8A-8B show the downregulated Tumor Necrosis Factor Alpha (TNF-α) levels in the brain tissue of the R6/2 transgenic HD mice treated by a Chinese herbal medicine composition of the present invention;

FIGS. 9A-9B show the downregulated levels of apoptosis-related indicator proteins in the brain tissue of the R6/2 transgenic HD mice treated by a Chinese herbal medicine composition of the present invention;

FIGS. 10A-10B show the enhanced microcirculation of the 3TgXAD transgenic Alzheimer's Disease (AD) mice treated by a Chinese herbal medicine composition of the present invention;

FIGS. 11A-11B show the upregulated Vascular Endothelial Growth Factor (VEGF) levels in the brain tissue of the 3TgXAD transgenic AD mice treated by a Chinese herbal medicine composition of the present invention;

FIGS. 12A-12B show the upregulated Brain-Derived Neurotrophic Factor (BDNF) levels in the brain tissue of the 3TgXAD transgenic AD mice treated by a Chinese herbal medicine composition of the present invention;

FIGS. 13A-13B show the downregulated Glycogen Synthase Kinase-3β (GSK-3β) levels in the brain tissue of the 3TgXAD transgenic AD mice treated by a Chinese herbal medicine composition of the present invention;

FIGS. 14A-14B show the downregulated phosphorylated Tau protein (p-Tau) levels in the brain tissue of the 3TgXAD transgenic AD mice treated by a Chinese herbal medicine composition of the present invention;

FIGS. 15A-15B show the downregulated neurofibrillary tangles (NTFs) levels in the brain tissue of the 3TgXAD transgenic AD mice treated by a Chinese herbal medicine composition of the present invention;

FIGS. 16A-16B show the downregulated amyloid beta (Aβ) levels in the brain tissue of the 3TgXAD transgenic AD mice treated by a Chinese herbal medicine composition of the present invention;

FIGS. 17A-17B show the downregulated superoxide dismutase 2 (SOD2) levels in the brain tissue of the 3TgXAD transgenic AD mice treated by a Chinese herbal medicine composition of the present invention;

FIGS. 18A-18B show the downregulated Tumor Necrosis Factor Alpha (TNF-α) levels in the brain tissue of the 3TgXAD transgenic AD mice treated by a Chinese herbal medicine composition of the present invention;

FIGS. 19A-19B show the improved memory of the 3TgXAD transgenic AD mice treated by a Chinese herbal medicine composition of the present invention;

FIGS. 20A-20B show the upregulated dopaminergic levels (DA) and dopaminergic receptor's (D₁R) levels in the brain tissue of the manganese-induced Parkinson's disease (PD) mice treated by a Chinese herbal medicine composition of the present invention;

FIGS. 21A-21B show the upregulated Brain-Derived Neurotrophic Factor (BDNF) levels in the brain tissue of the manganese-induced PD mice treated by a Chinese herbal medicine composition of the present invention;

FIGS. 22A-22B show the upregulated of Reactive Oxygen Species (ROS) in the blood of the manganese-induced PD mice treated by a Chinese herbal medicine composition of the present invention; and

FIG. 23 show the improved motor coordination of the manganese-induced PD mice treated by a Chinese herbal medicine composition of the present invention.

### DETAILED DESCRIPTION OF THE PREFERRED EMBODIMENT

### Preparation Example

A Chinese herbal medicine composition is provided, which comprises a first herbal formula and a second herbal formula. The first herbal formula comprises 2-40 parts by weight of Panax ginseng and 0.8-20 parts by weight of Angelica sinensis. The second herbal formula comprises 1-24 parts by weight of Angelica sinensis, 2-40 parts by weight of Astragalus membranaceus, and 20-96 parts by weight of a mixture of Ligustri fructus, Eclipta prostrata, and Rehmannia glutinosa. A weight ratio of Ligustri fructus, Eclipta prostrata, and Rehmannia glutinosa in the mixture is 0.8-1.2:0.8-1.2:0.8-1.2, and more preferably, the weight ratio of Ligustri fructus, Eclipta prostrata, and Rehmannia glutinosa in the mixture is 1:1:1.

First, the first herbal formula is extracted using an alcohol with a concentration of 60-70 Vol %. A first extract is obtained after filtering the solids without efficacy of the first herbal formula by centrifugation. The second herbal formula is extracted by decocting with boiling water to obtain a crude extract decoction. A second extract is obtained after filtering the solids without efficacy of the second herbal formula by centrifugation. A third extract is obtained after the second extract is filtered using ceramic membranes.

A Chinese herbal medicine composition of the present invention is formed after the first extract and the third extract are mixed.

### Experiment Example 1

### SH-SY5Y Neuronal Differentiation Cell Model

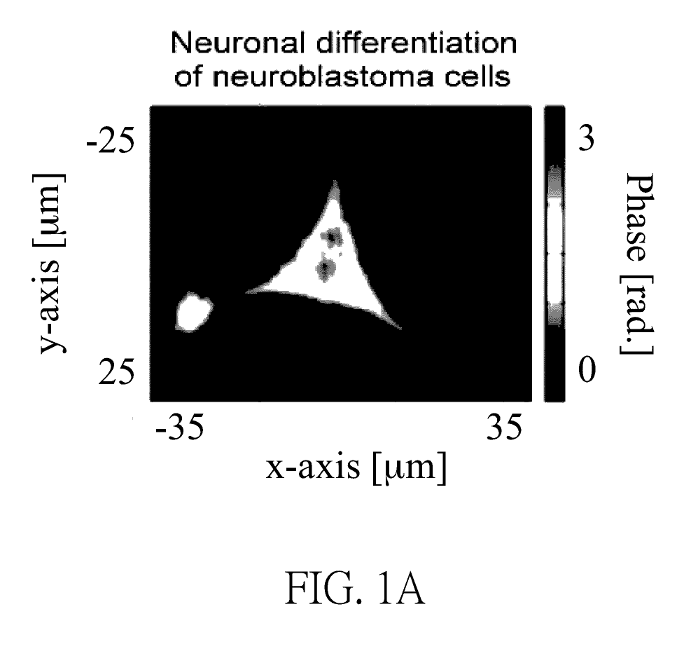

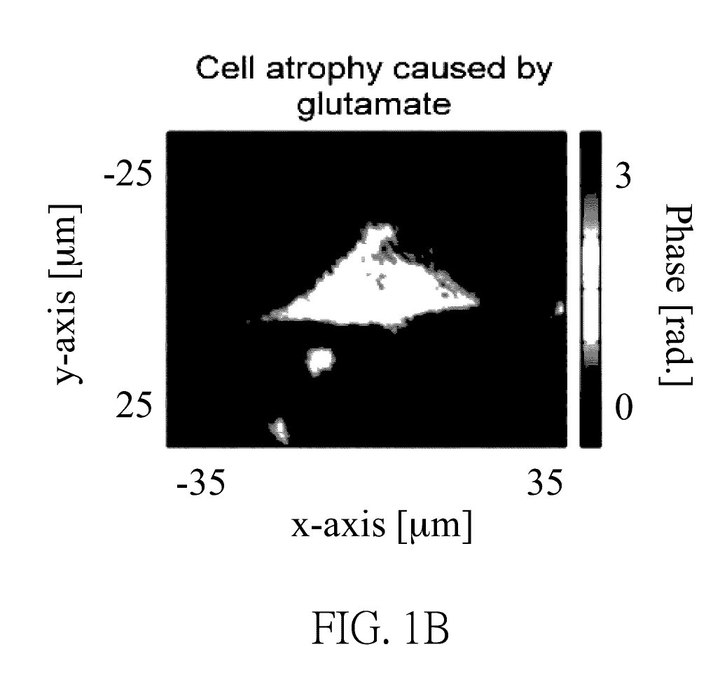

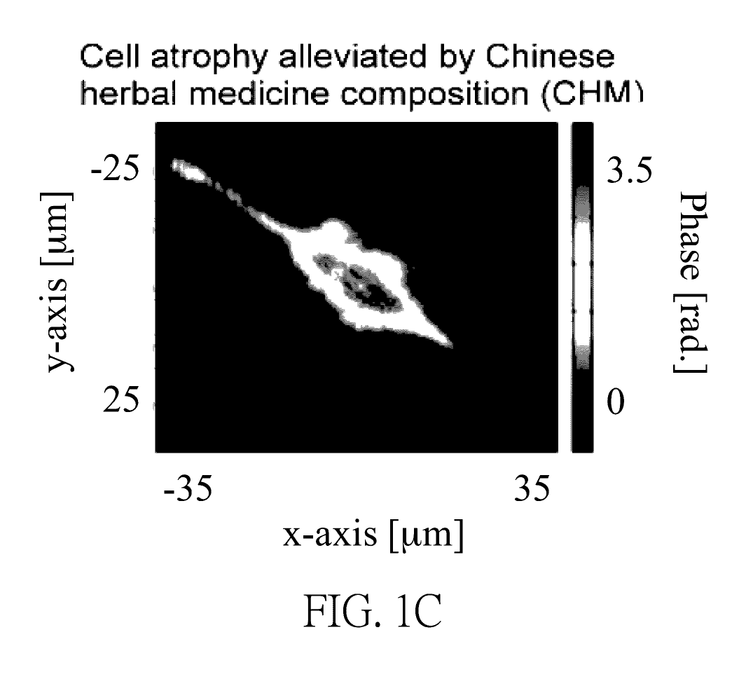

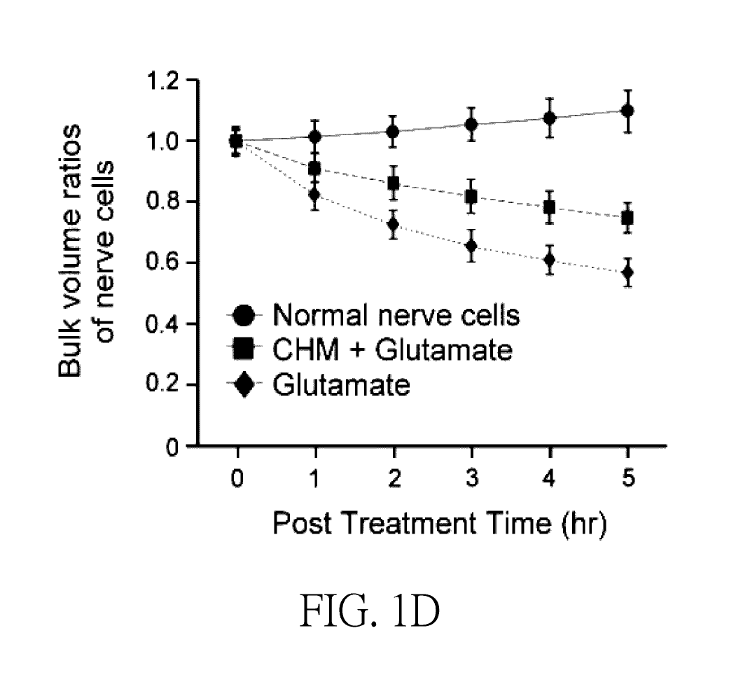

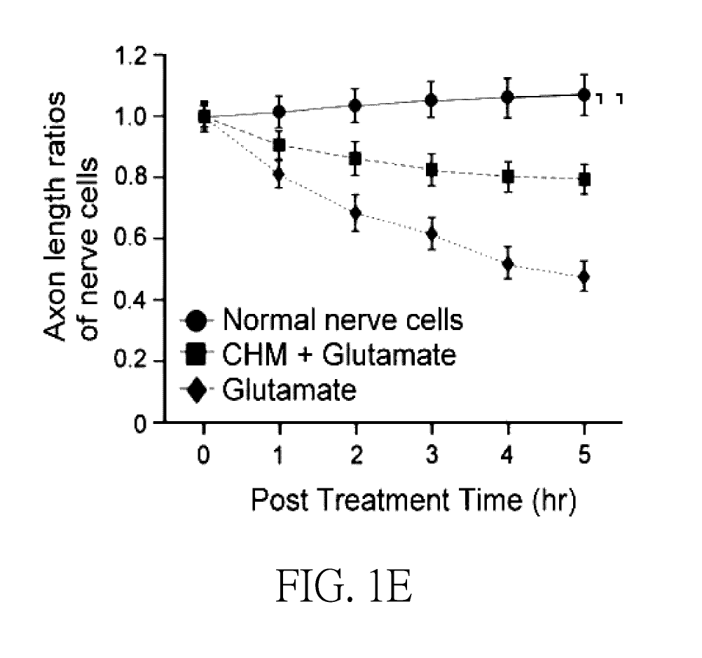

Please refer to FIGS. 1A-1C, retinoic acid (RA) is first added to SH-SY5Y neuroblastoma cells to induce neuronal differentiation. Glutamate is then added to initiate excitotoxic killing, causing cell atrophy. When the Chinese herbal medicine composition (CHM) (30 mM) of the present invention is added, cell atrophy caused by glutamate has slowed down effectively.

Please refer to FIGS. 1B-1C. FIGS. 1B-1C show the relationship between volume change and time of normal nerve cells, nerve cells pretreated with the Chinese herbal medicine composition of the present invention for 12 hours and then treated with glutamate, and nerve cells treated with glutamate to initiate excitotoxic killing of cells. As shown in FIGS. 1B-1C, cell atrophy caused by the excitotoxic killing initiated by glutamate can be delayed effectively by the pre-treatment of the Chinese herbal medicine composition of the present invention (p<0.01, N=12 per group). In other words, the pre-treatment of the Chinese herbal medicine composition of the present invention has a preventive effect.

### Experiment Example 2

### Huntington's Disease (HD) Model

Huntington's Disease R6/2 transgenic mice (B6CBA-Tg(HD exon1)65Gpb/1J) (HD mice) are used as test subjects. The neurological phenotypes of these HD mice are similar to the symptoms of Huntington's disease of human beings. For example, the neurological phenotypes of these HD mice progress with age, including choreiform-like or unintentional movements, tremor, and epilepsy. These HD mice may also suffer from muscle wasting and premature death. In addition, huntingin protein could also be found in neuronal intranuclear inclusions of these HD mice. Since the symptoms of Huntington's diseases of these HD mice begin at 8 weeks old and these HD mice begin to die at 14 weeks old, early Huntington's disease treatment is performed using HD mice of two months old and late Huntington's disease treatment is performed using HD mice of three months old.

Specifically, 2-month R6/2 HD mice are used for oral treatment with either the Chinese herbal medicine composition of the present invention (30 mg/mL) or a control in the drinking water. The control only comprises solvents used for extraction and does not comprise the Chinese herbal medicine composition of the present invention. All doses are adjusted according to individual weight and water consumption, which are an average of 30 mL/day. On the other hand, 3-month R6/2 HD mice are used for intraperitoneal injection of either the Chinese herbal medicine composition of the present invention or a control. The control only comprises solvents used for extraction in saline solution and does not comprise the Chinese herbal medicine composition of the present invention. All doses are adjusted according to individual weight, which are an average of 10 mL/day.

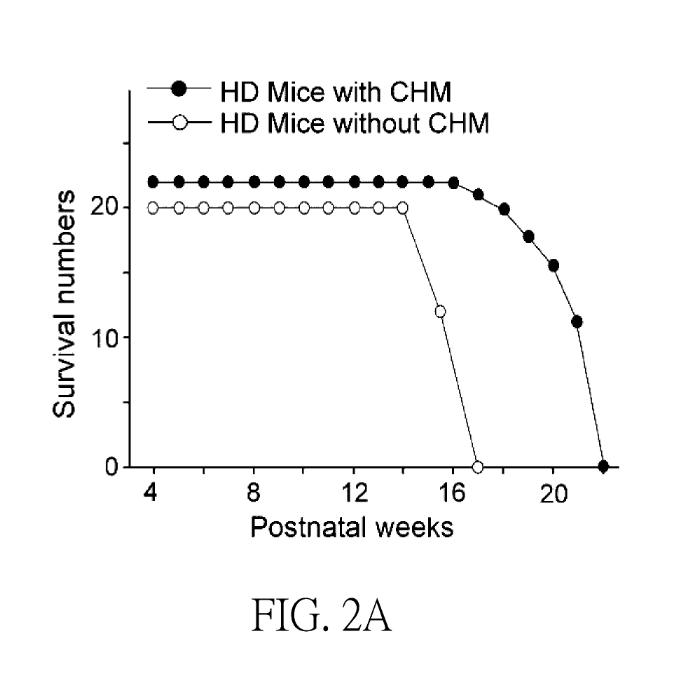

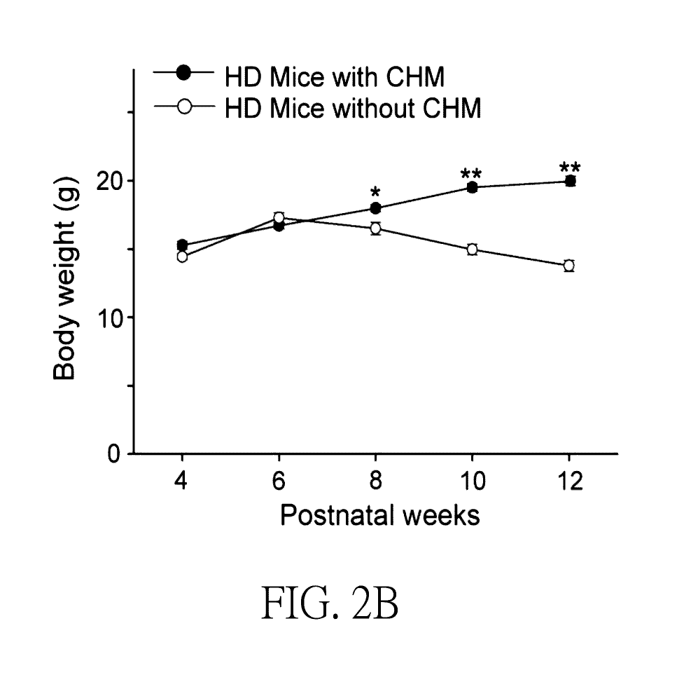

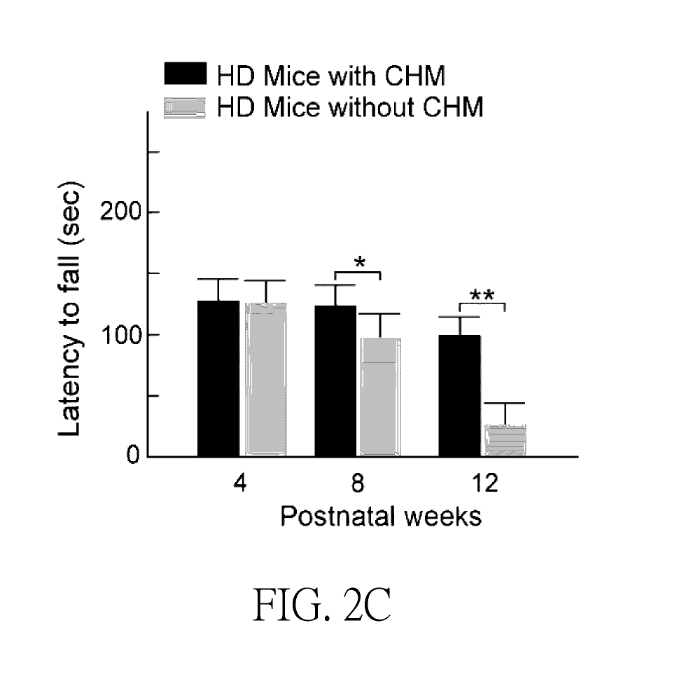

As shown in FIG. 2A, the survival duration of HD mice (22 mice) fed with the Chinese herbal medicine composition of the present invention is longer than HD mice (22 mice) unfed with the Chinese herbal medicine composition of the present invention. As shown in FIG. 2B, the body weight of HD mice (22 mice) fed with the Chinese herbal medicine composition of the present invention is significantly heavier than HD mice (22 mice) unfed with the Chinese herbal medicine composition of the present invention. FIG. 2C shows the motor performance of 3-month HD mice tested using rotarod testing. As shown in FIG. 2C, the latencies to fall of HD mice (12 mice) fed with the Chinese herbal medicine composition of the present invention is significantly longer than the HD mice (12 mice) unfed with the Chinese herbal medicine composition of the present invention (p<0.01-0.05).

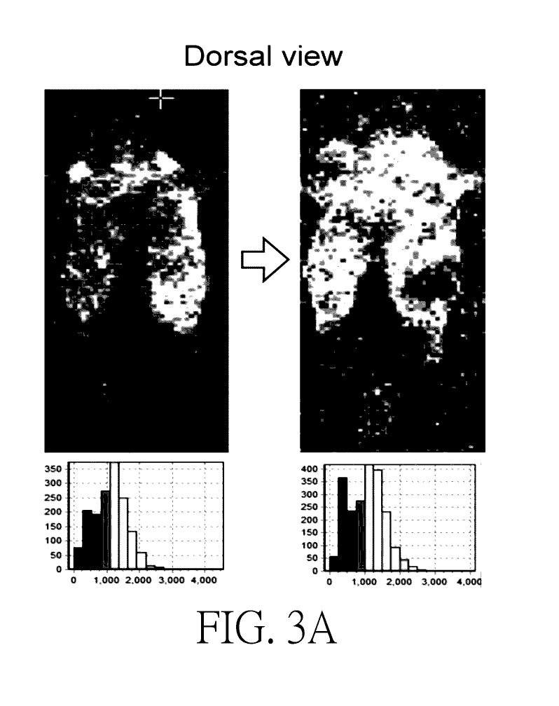

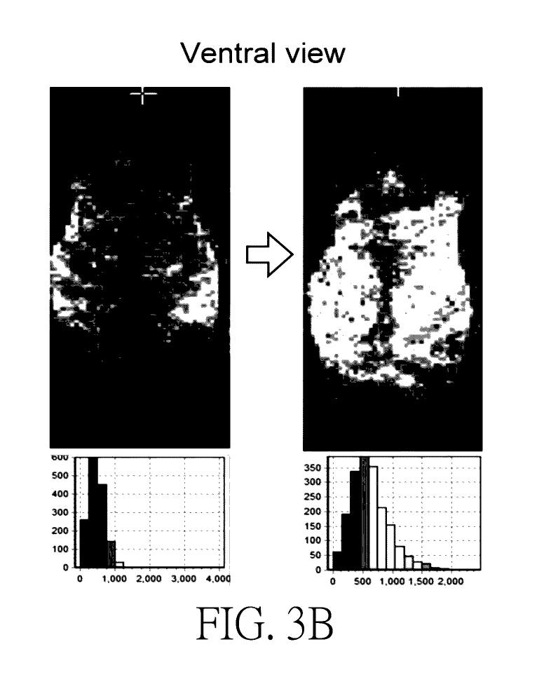

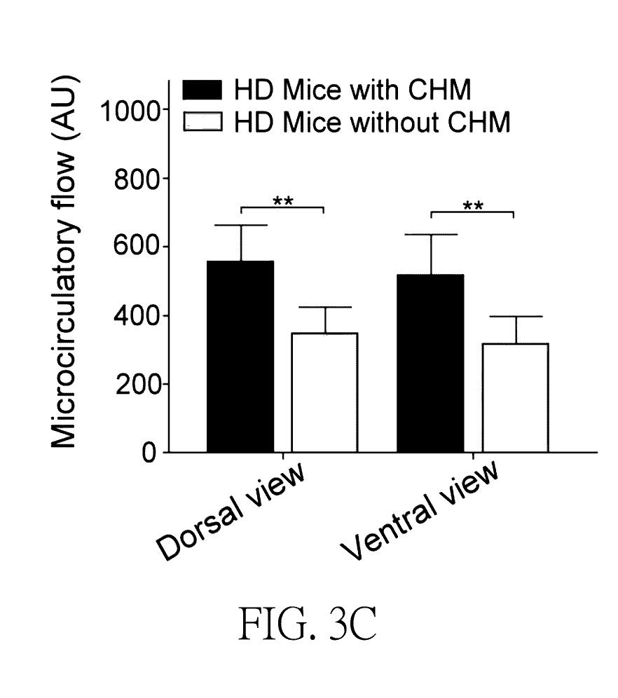

FIGS. 3A-3B show the dorsal and ventral subcutaneous microcirculatory flows of 3-month HD mice before the injection (at the left of the arrow) or after the injection (at the right of the arrow) of the Chinese herbal medicine composition of the present invention using Moor LDI2 high-resolution laser Doppler imager. As shown in FIGS. 3A-3B, the dorsal and ventral subcutaneous microcirculatory flows of HD mice increase after the injection (at the right of the arrow) of the Chinese herbal medicine composition of the present invention. FIG. 3C shows the quantifications of the dorsal and ventral subcutaneous microcirculatory flows of HD mice after the injection (at the right of the arrow) of the Chinese herbal medicine composition of the present invention. As shown in FIG. 3C, the dorsal and ventral subcutaneous microcirculatory flows of HD mice increase significantly after the injection (at the right of the arrow) of the Chinese herbal medicine composition of the present invention (p<0.01, 6 HD mice per group).

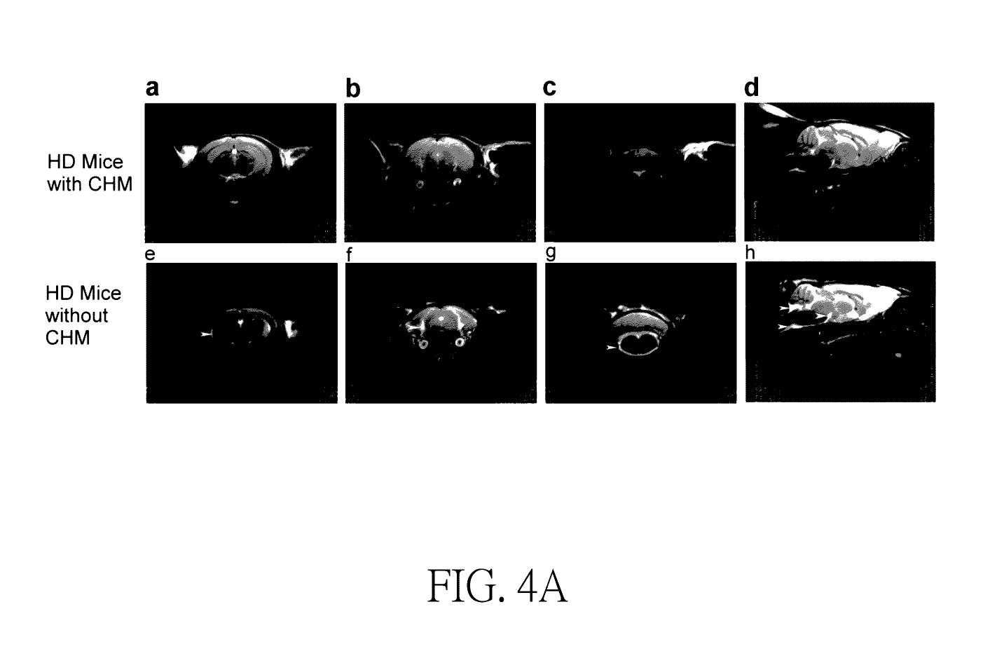

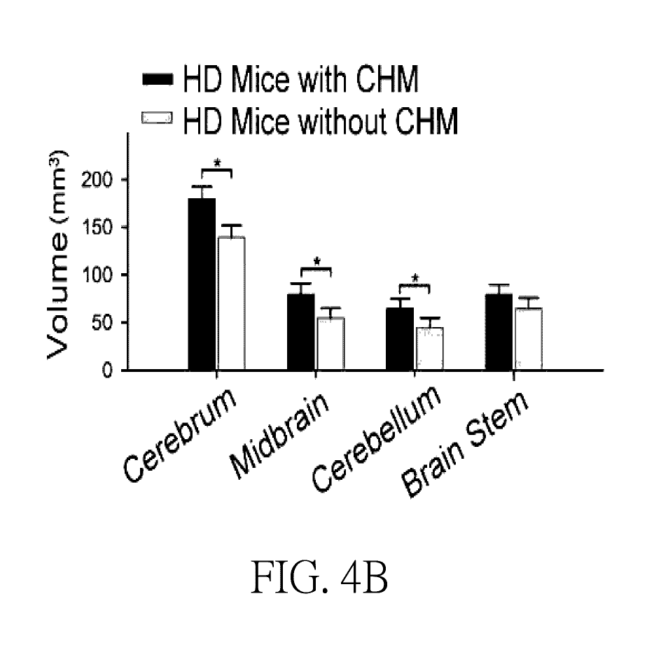

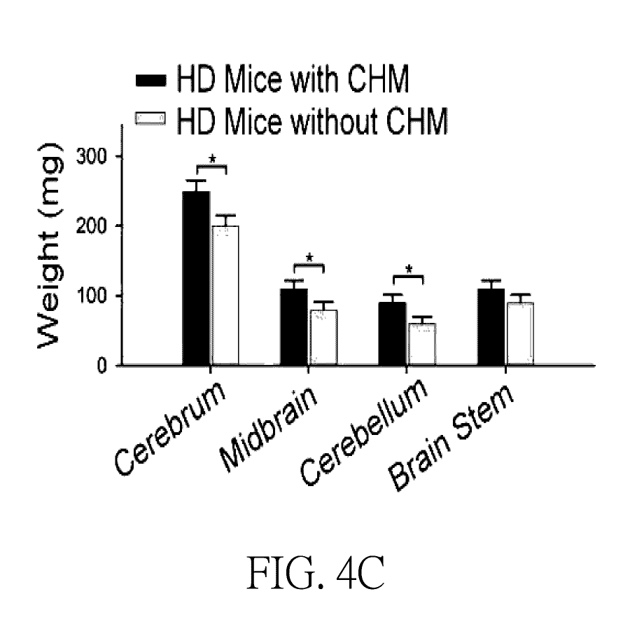

FIG. 4A shows the brain structure of 3-month HD mice studied by magnetic resonance image (MRI). As shown in FIG. 4A, visible atrophy in the brain of HD mice is observed and this atrophy is a progressive, region-specific, and age-dependent brain atrophy. However, brain atrophy of HD mice fed with the Chinese herbal medicine composition of the present invention is alleviated because both volume and weight of cerebrum, midbrain, and cerebellum of the HD mice fed with the Chinese herbal medicine composition of the present invention are significantly greater than the HD mice unfed with the Chinese herbal medicine composition of the present invention. FIGS. 4B-4C show the quantification of the volume and weight of the brain of HD mice. As shown in FIGS. 4B-4C, both volume and weight of cerebrum, midbrain, and cerebellum of the HD mice fed with the Chinese herbal medicine composition of the present invention are significantly greater than the HD mice unfed with the Chinese herbal medicine composition of the present invention (p<0.05, 6 HD mice per group).

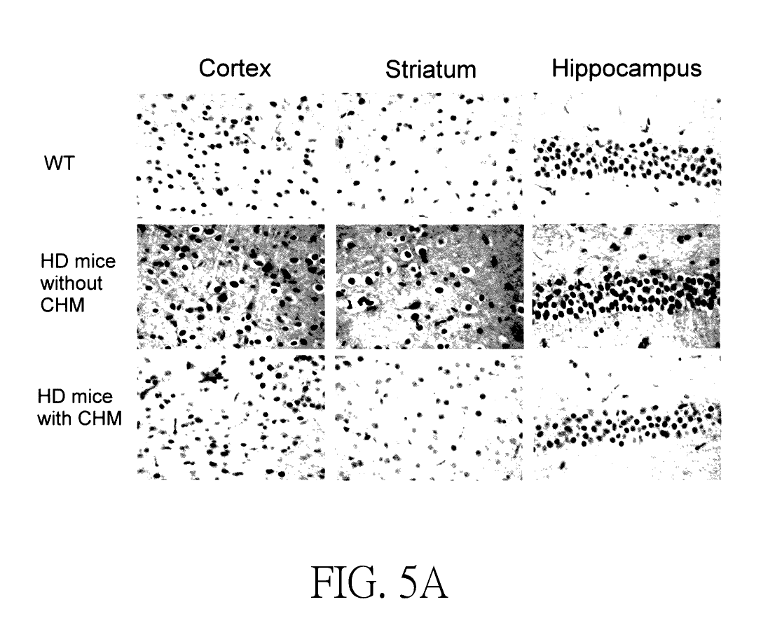

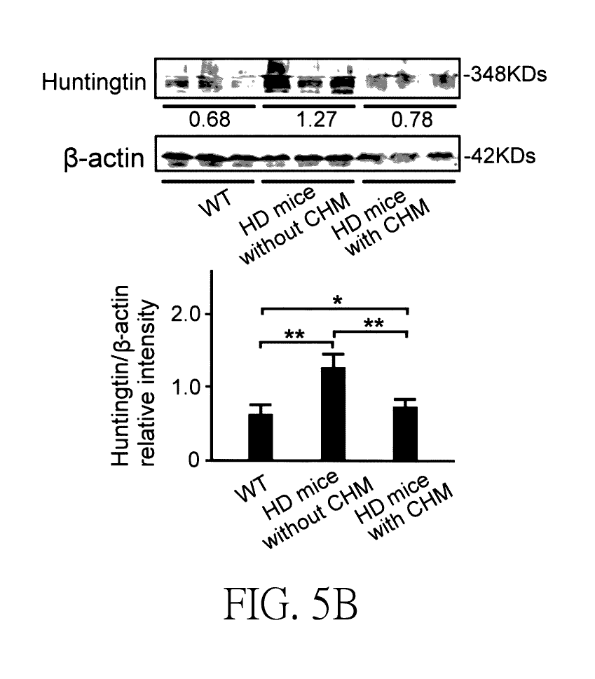

Since the deposition of mutant huntingtin is a neuropathological hallmark of Huntington's disease, whether the mutant huntingtin level in the brain can be reduced by the Chinese herbal medicine composition of the present invention is determined. FIGS. 5A-5B show the levels of the mutant huntingtin in the brains of 2-month HD mice treated with or without the Chinese herbal medicine composition of the present invention and wild type (WT) littermates (6 mice per group). As shown in FIG. 5A, the expression levels of mutant huntingtin in the cortex, striatum, and hippocampus of HD mice treated with the Chinese herbal medicine composition of the present invention are reduced (as shown by immunohistochemical staining). As shown in FIG. 5B, the expression levels of mutant huntingtin in the brain tissues of HD mice treated with the Chinese herbal medicine composition of the present invention are reduced (as shown by western blotting) (p<0.01-0.05).

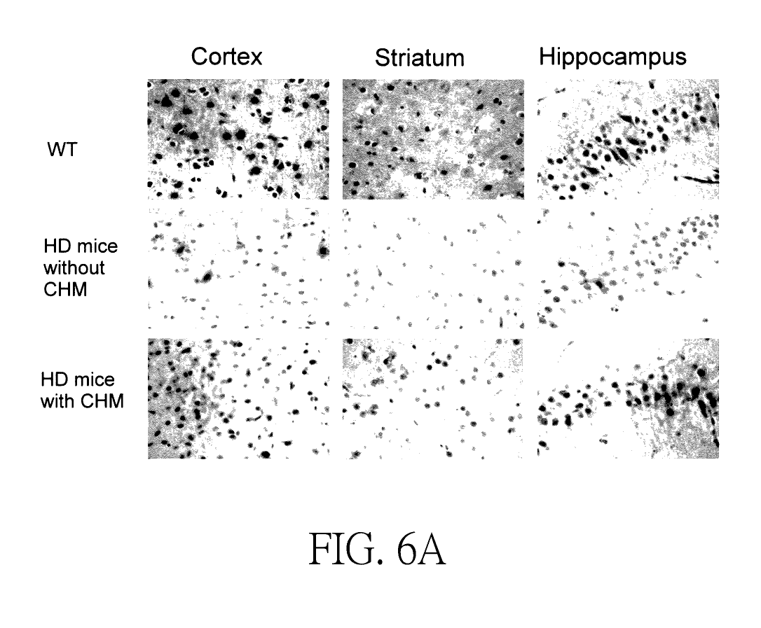

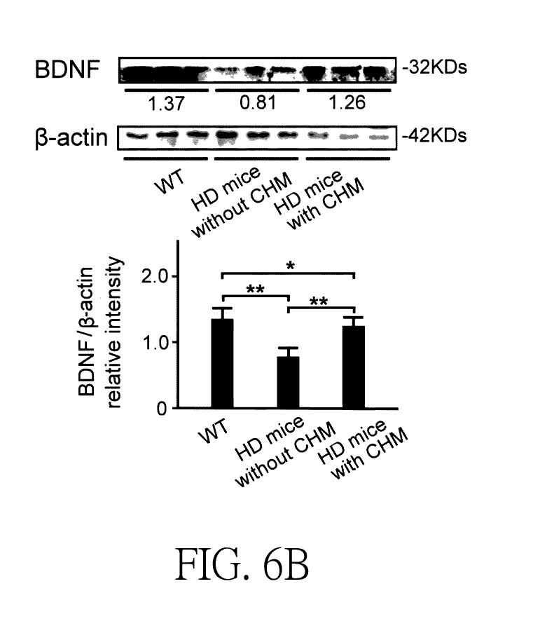

Since the neurotrophic hypothesis of Huntington's disease proposes that impaired production of Brain-Derived Neurotrophic Factor (BDNF) contributes to degeneration of neurons in the striatum, whether the expression level of BDNF in the brain can be increased by the Chinese herbal medicine composition of the present invention is determined. FIGS. 6A-6B show the expression levels of BDNF in the brains of 2-month HD mice treated with or without the Chinese herbal medicine composition of the present invention and wild type (WT) littermates (6 mice per group). As shown in FIG. 6A, the expression levels of BDNF in the cortex, striatum, and hippocampus of HD mice treated with the Chinese herbal medicine composition of the present invention are increased (as shown by immunohistochemical staining). As shown in FIG. 6B, the expression levels of BDNF in the brain tissues of HD mice treated with the Chinese herbal medicine composition of the present invention are significantly enhanced (as shown by western blotting) (p<0.01-0.05).

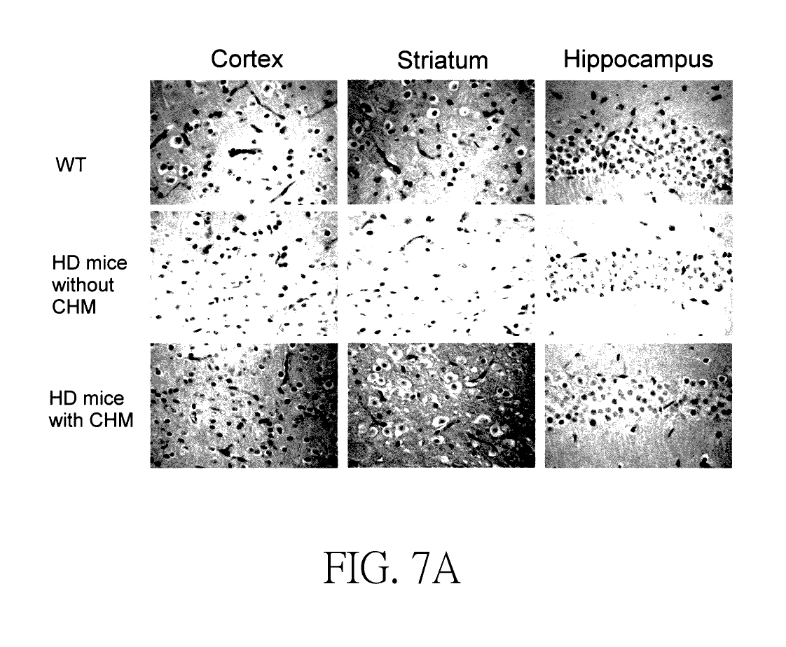

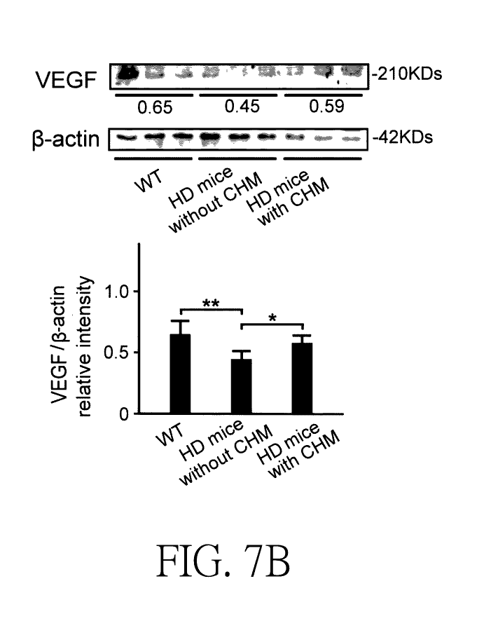

Since Vascular Endothelial Growth Factor (VEGF) in the brain plays a crucial role in neuroprotective and angiogenic effects, whether the expression level of VEGF in the brain can be enhanced by the Chinese herbal medicine composition of the present invention is determined. FIGS. 7A-7B show the expression levels of VEGF in the brains of 2-month HD mice treated with or without the Chinese herbal medicine composition of the present invention and wild type (WT) littermates (6 mice per group). As shown in FIG. 7A, the expression levels of VEGF in the cortex, striatum, and hippocampus of HD mice treated with the Chinese herbal medicine composition of the present invention are enhanced (as shown by immunohistochemical staining). As shown in FIG. 7B, the expression levels of VEGF in the brain tissues of HD mice treated with the Chinese herbal medicine composition of the present invention are significantly enhanced (as shown by western blotting) (p<0.01-0.05).

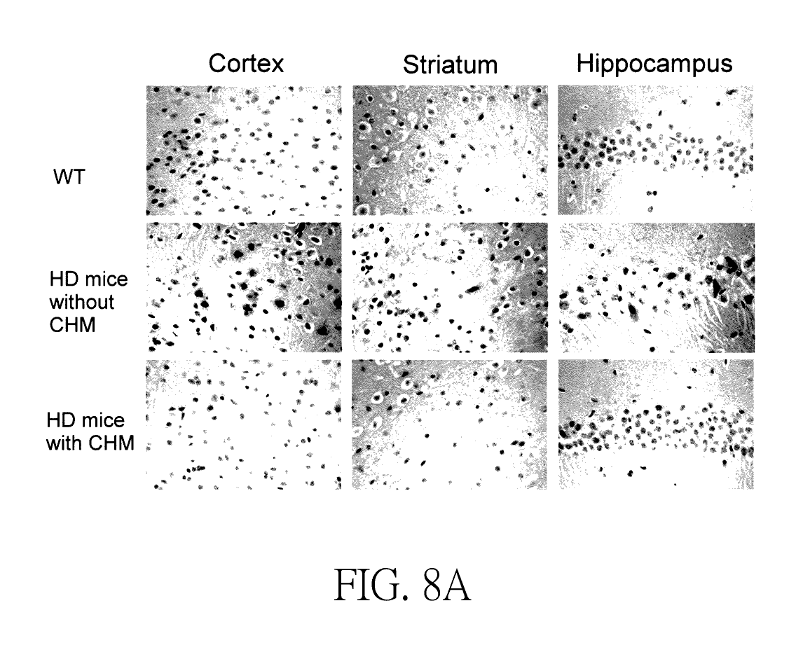

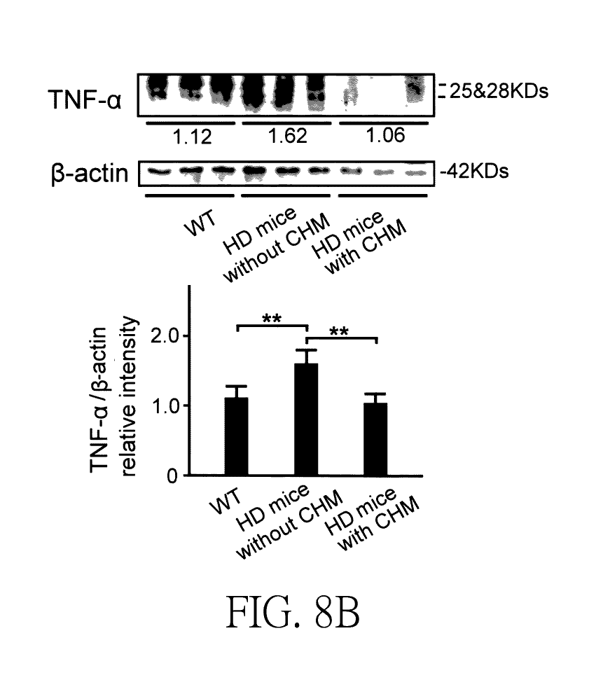

Since inflammation is believed to be involved in the pathogenesis and progression of neurologic diseases and Tumor Necrosis Factor Alpha (TNF-α) is a key marker for inflammation, whether the expression level of TNF-α in the brain can be reduced by the Chinese herbal medicine composition of the present invention is determined. FIGS. 8A-8B shows the expression levels of TNF-α in the brains of 2-month HD mice treated with or without the Chinese herbal medicine composition of the present invention and wild type (WT) littermates (6 mice per group). As shown in FIG. 8A, the expression levels of TNF-α in the cortex, striatum, and hippocampus of HD mice treated with the Chinese herbal medicine composition of the present invention are reduced (as shown by immunohistochemical staining). As shown in FIG. 8B, the expression levels of TNF-α in the brain tissues of HD mice treated with the Chinese herbal medicine composition of the present invention are significantly reduced (as shown by western blotting) (p<0.01).

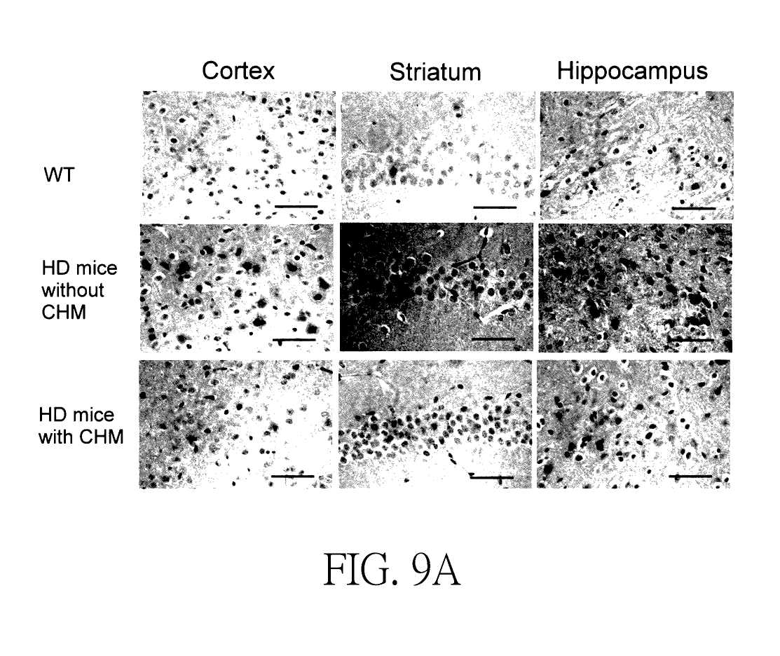

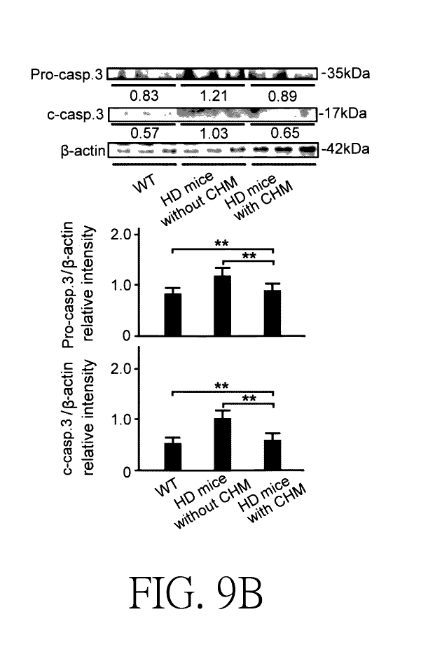

The function of mitochondria is confirmed next. Since Caspase-3 is an indicator protein related to abnormal mitochondria function (cell apoptosis), whether the expression level of Caspase-3 in the brain can be reduced by the Chinese herbal medicine composition of the present invention is determined. FIGS. 9A-9C show the expression levels of Caspase-3 in the brains of 2-month HD mice treated with or without the Chinese herbal medicine composition of the present invention and wild type (WT) littermates (6 mice per group). As shown in FIG. 9A, the expression levels of Caspase-3 in the cortex, striatum, and hippocampus of HD mice treated with the Chinese herbal medicine composition of the present invention are reduced (as shown by immunohistochemical staining). As shown in FIG. 9B, the expression levels of Caspase-3 in the brain tissues of HD mice treated with the Chinese herbal medicine composition of the present invention are significantly reduced (as shown by western blotting) (p<0.01).

As shown by the results of Experiment Example 2 described above, the Chinese herbal medicine composition of the present invention can down-regulate the deposition of mutant huntingtin in the brain. In addition, by reducing the expression level of TNF-α, excitotoxic neurodegeneration diseases can be reduced, inflammation can be alleviated, and cell apoptosis caused by abnormal mitochondria function can be reduced. On the other hand, the Chinese herbal medicine composition of the present invention can promote expression levels of BDNF and VEGF in the brain to provide protective effect of the brain. Accordingly, the Chinese herbal medicine composition of the present invention has a great potential as a drug to treat or suppress Huntington's disease.

### Experiment Example 3

### Alzheimer's Disease (AD) Model

In Experiment Example 3, the effect of the Chinese herbal medicine composition of the present invention in the treatment or prevention of Alzheimer's disease is explored. Alzheimer's Disease 3TgXAD transgenic mice (human APPswe×human PS1M146V×human tauP301L; 3×Tg-AD) (AD mice) are used as test subjects. The life cycle of these AD mice is approximately 18 months. Since the onset of disease of these AD mice is at nine months old, AD mice of ten months old showing symptoms of Alzheimer's disease are treated with the Chinese herbal medicine composition of the present invention prepared in the Preparation Example for two months.

From current researches, it is generally agreed that when the energy of brain, such as glucose and oxygen, is insufficient, deterioration of dementia will be accelerated.

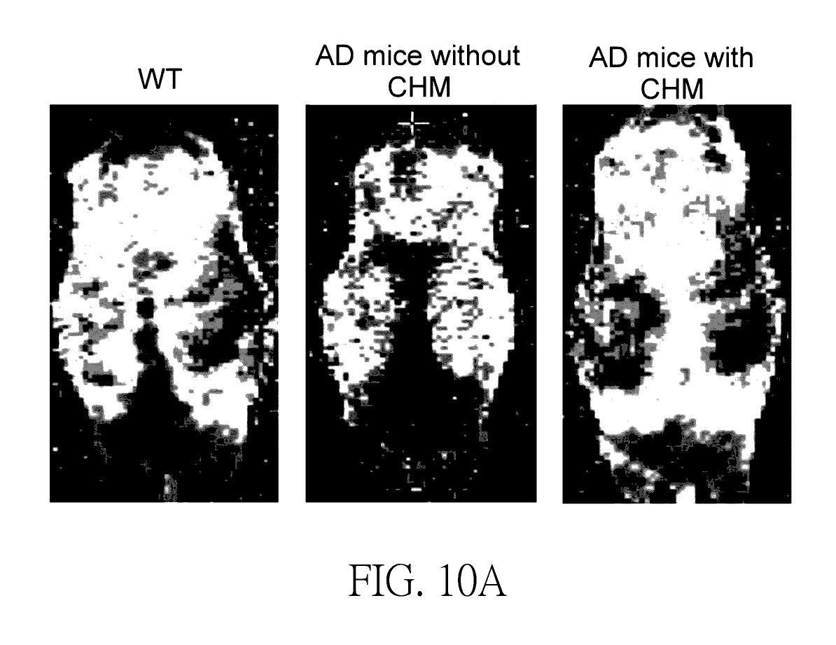

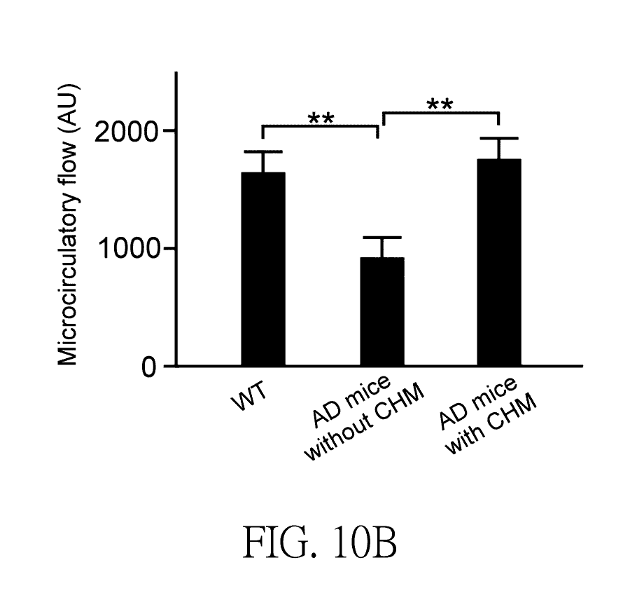

FIG. 10A shows the subcutaneous microcirculatory flows of 12-month AD mice treated with or without the Chinese herbal medicine composition of the present invention and Wild type (WT) littermates using Moor LDI2 high-resolution laser Doppler imager. As shown in FIG. 10A, the dorsal subcutaneous microcirculatory flows of AD mice treated with the Chinese herbal medicine composition of the present invention have increased. FIG. 10B shows the quantifications of the dorsal subcutaneous microcirculatory flows of AD mice treated with the Chinese herbal medicine composition of the present invention. As shown in FIG. 10B, the dorsal subcutaneous microcirculatory flows of AD mice treated with the Chinese herbal medicine composition of the present invention have significantly increased (p<0.01, 6 AD mice per group).

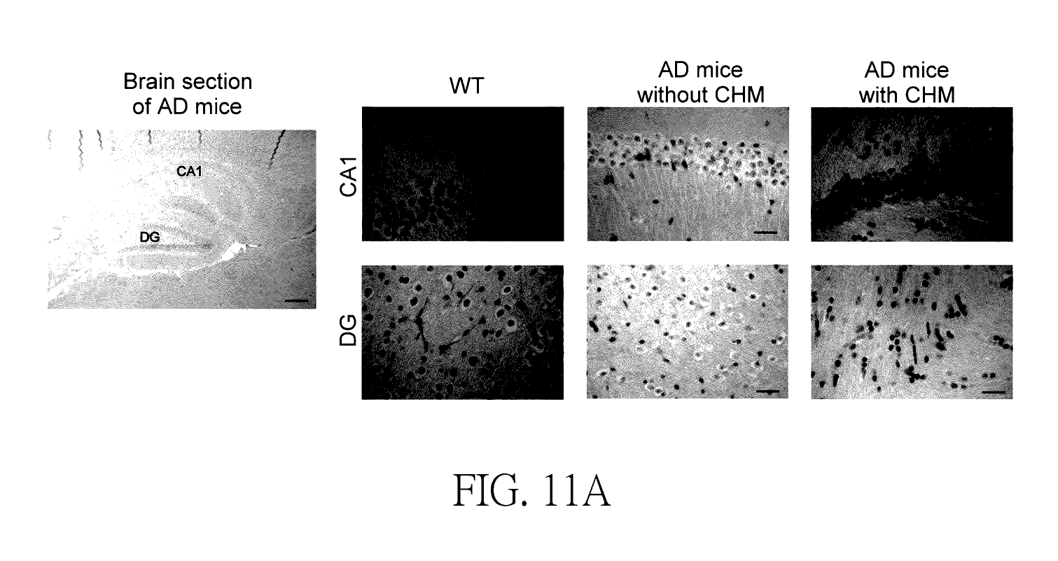

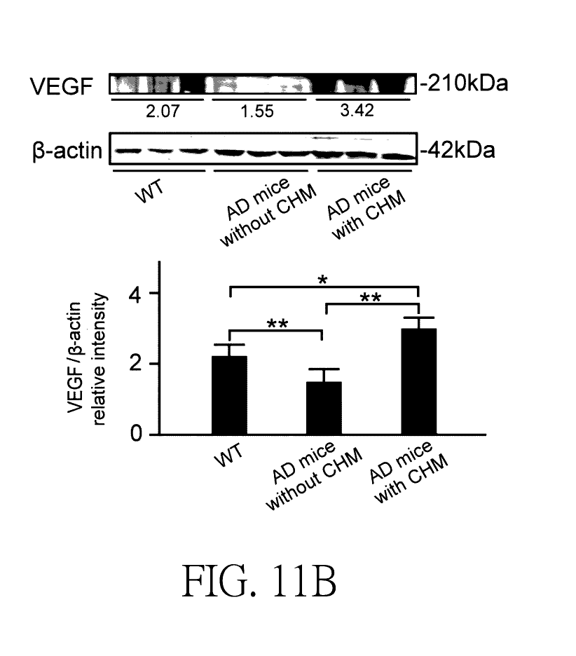

Since Vascular Endothelial Growth Factor (VEGF) in the brain plays a crucial role in neuroprotective and angiogenic effects, whether the expression level of VEGF in the brain can be enhanced by the Chinese herbal medicine composition of the present invention is determined. FIGS. 11A-11B show the expression levels of VEGF in the brains of 9-month AD mice treated with or without the Chinese herbal medicine composition of the present invention and wild type (WT) littermates (6 mice per group). As shown in FIG. 11A, the expression levels of VEGF in the hippocampal CA1 region (CA1) and the dentate gyrus (DG) of AD mice treated with the Chinese herbal medicine composition of the present invention are enhanced (as shown by immunohistochemical staining). As shown in FIG. 11B, the expression levels of VEGF in the brain tissues of AD mice treated with the Chinese herbal medicine composition of the present invention are significantly enhanced (as shown by western blotting) (p<0.01-0.05).

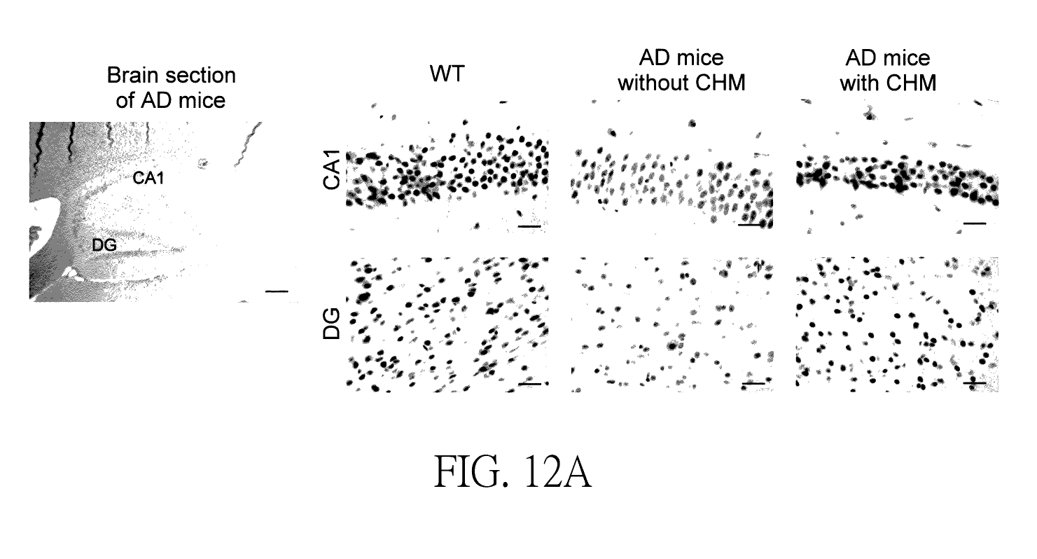

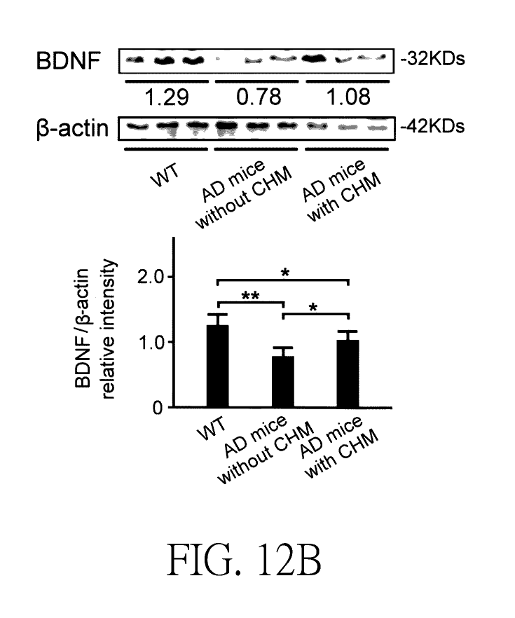

FIGS. 12A-12B show the expression levels of Brain-Derived Neurotrophic Factor (BDNF) in the brains of 9-month AD mice treated with or without the Chinese herbal medicine composition of the present invention and wild type (WT) littermates (6 mice per group). As shown in FIG. 12A, the expression levels of BDNF in the hippocampal CA1 region (CA1) and the dentate gyrus (DG) of AD mice treated with the Chinese herbal medicine composition of the present invention are increased (as shown by immunohistochemical staining). As shown in FIG. 12B, the expression levels of BDNF in the brain tissues of AD mice treated with the Chinese herbal medicine composition of the present invention are significantly enhanced (as shown by western blotting) (p<0.01-0.05).

As shown by the results described above, when the AD mice are treated with the Chinese herbal medicine composition of the present invention, microcirculatory flow is increased, microvessel growth is promoted, and permeability is increased. Consequently, blood circulation is increased to provide nutrients needed by the brain more efficiently, improving memory. The Chinese herbal medicine composition of the present invention can also promote expression levels of BDNF and VEGF in the brain to provide protective effect of the brain, reducing damages of nerve cells.

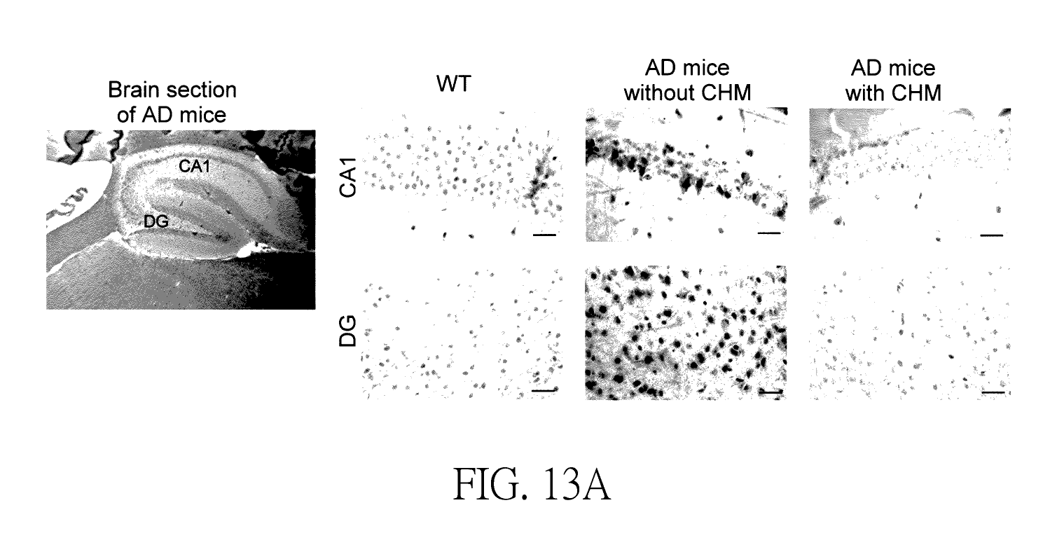

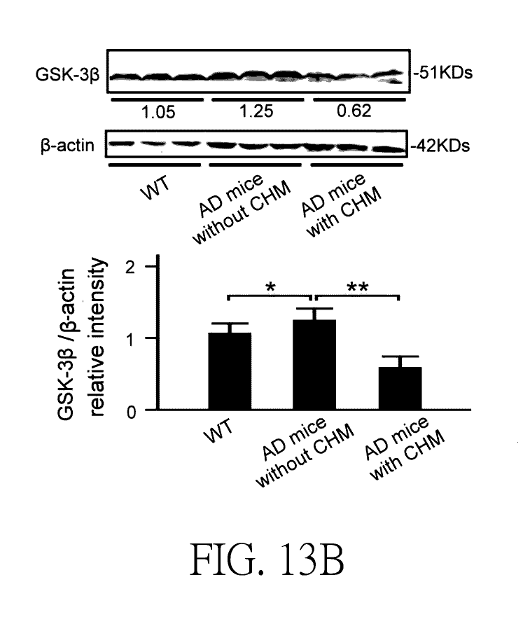

Glycogen Synthase Kinase-3β (GSK-3β) is one of the important Tau protein kinases in the pathway of Tau protein aggregation of Alzheimer's disease. It can catalyze the phosphorylation of Tau protein at multiple sites. The neurotrophic factor BDNF can suppress the activity of GSK-3β. As a result, whether the expression level of GSK-3β in the brain can be reduced by the Chinese herbal medicine composition of the present invention is determined. FIGS. 13A-13B show the expression levels of GSK-3β in the brains of 9-month AD mice treated with or without the Chinese herbal medicine composition of the present invention and wild type (WT) littermates (6 mice per group). As shown in FIG. 13A, the expression levels of GSK-31:3 in the hippocampal CA1 region (CA1) and the dentate gyrus (DG) of AD mice treated with the Chinese herbal medicine composition of the present invention are reduced (as shown by immunohistochemical staining). As shown in FIG. 13B, the expression levels of GSK-3β in the brain tissues of AD mice treated with the Chinese herbal medicine composition of the present invention are significantly reduced (as shown by western blotting) (p<0.01-0.05).

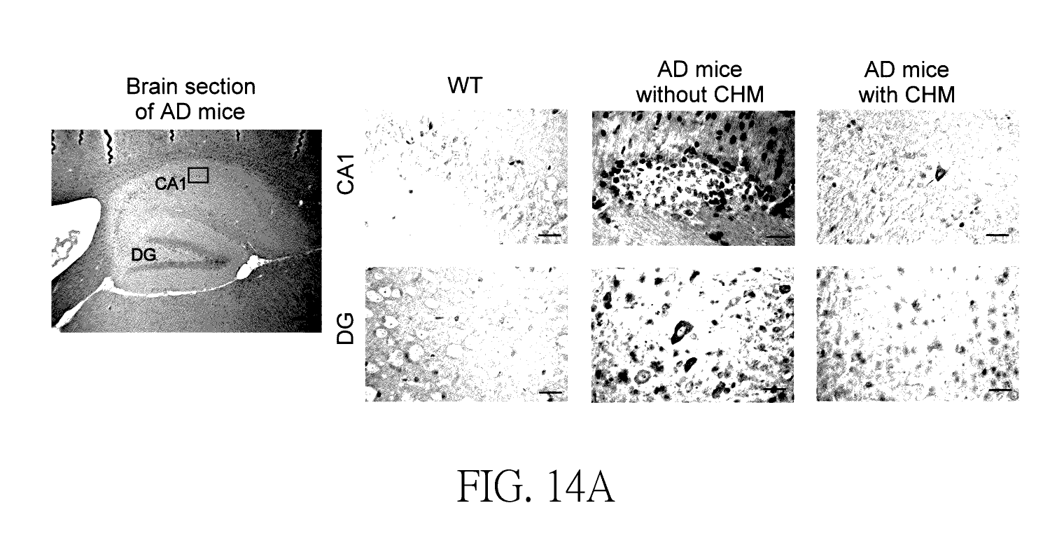

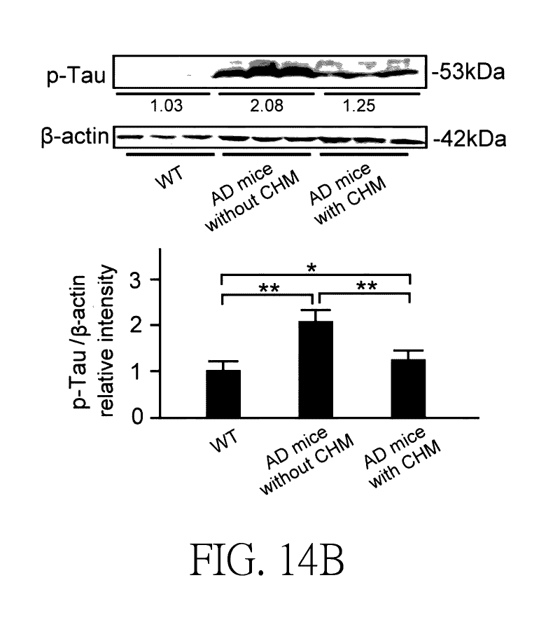

Since GSK-3β can catalyze the phosphorylation of Tau protein at multiple sites and the Chinese herbal medicine composition of the present invention can significantly reduce the activity of GSK-3β, whether the expression level of phosphorylated Tau protein (p-Tau) in the brain can be reduced by the Chinese herbal medicine composition of the present invention is determined. FIGS. 14A-14B show the expression levels of p-Tau in the brains of 9-month AD mice treated with or without the Chinese herbal medicine composition of the present invention and wild type (WT) littermates (6 mice per group). As shown in FIG. 14A, the expression levels of p-Tau in the hippocampal CA1 region (CA1) and the dentate gyrus (DG) of AD mice treated with the Chinese herbal medicine composition of the present invention are reduced (as shown by immunohistochemical staining). As shown in FIG. 14B, the expression levels of p-Tau in the brain tissues of AD mice treated with the Chinese herbal medicine composition of the present invention are significantly reduced (as shown by western blotting) (p<0.01-0.05).

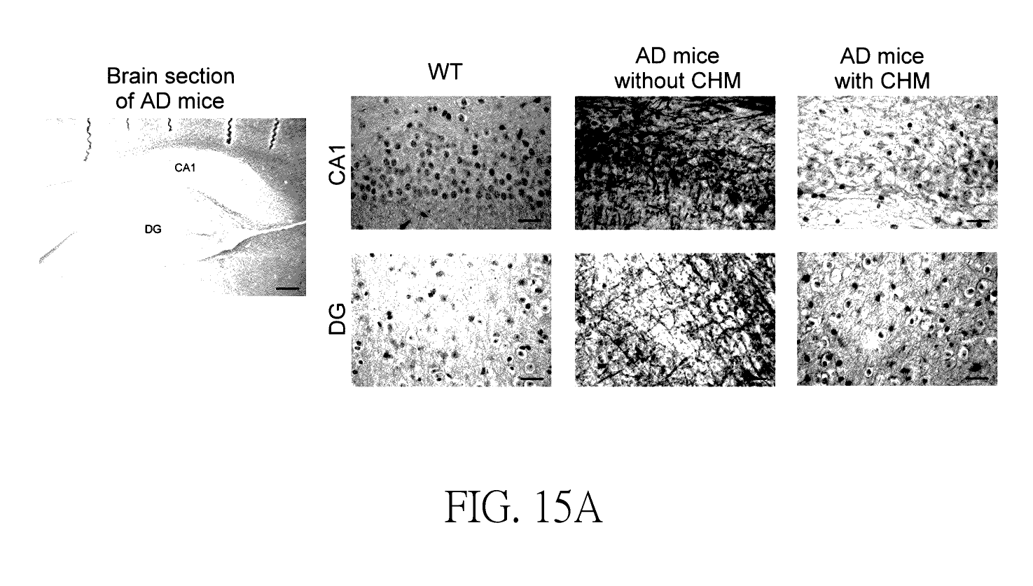

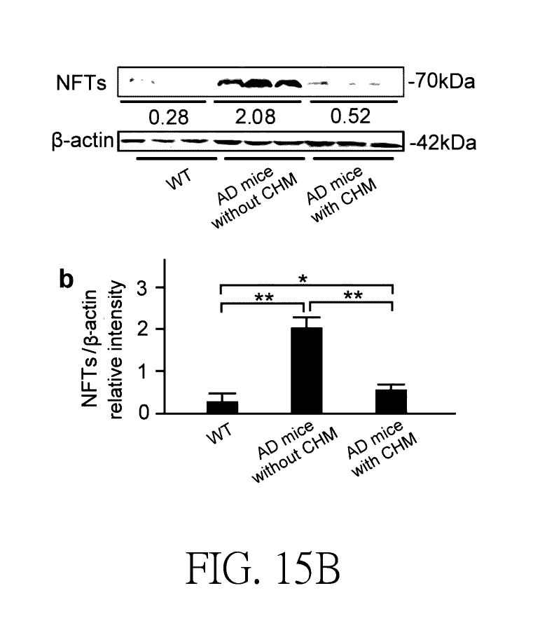

As suggested by past studies, excessive activation of GSK-3β can cause hyperphosphorylation of Tau protein, leading to Neurofibrillary Tangles (NTFs). As a result, whether the expression level of NTFs in the brain can be reduced by the Chinese herbal medicine composition of the present invention is determined. FIGS. 15A-15B show the expression levels of NTFs in the brains of 9-month AD mice treated with or without the Chinese herbal medicine composition of the present invention and wild type (WT) littermates (6 mice per group). As shown in FIG. 15A, the expression levels of NTFs in the hippocampal CA1 region (CA1) and the dentate gyrus (DG) of AD mice treated with the Chinese herbal medicine composition of the present invention are reduced (as shown by immunohistochemical staining). As shown in FIG. 15B, the expression levels of NTFs in the brain tissues of AD mice treated with the Chinese herbal medicine composition of the present invention are significantly reduced (as shown by western blotting) (p<0.01-0.05).

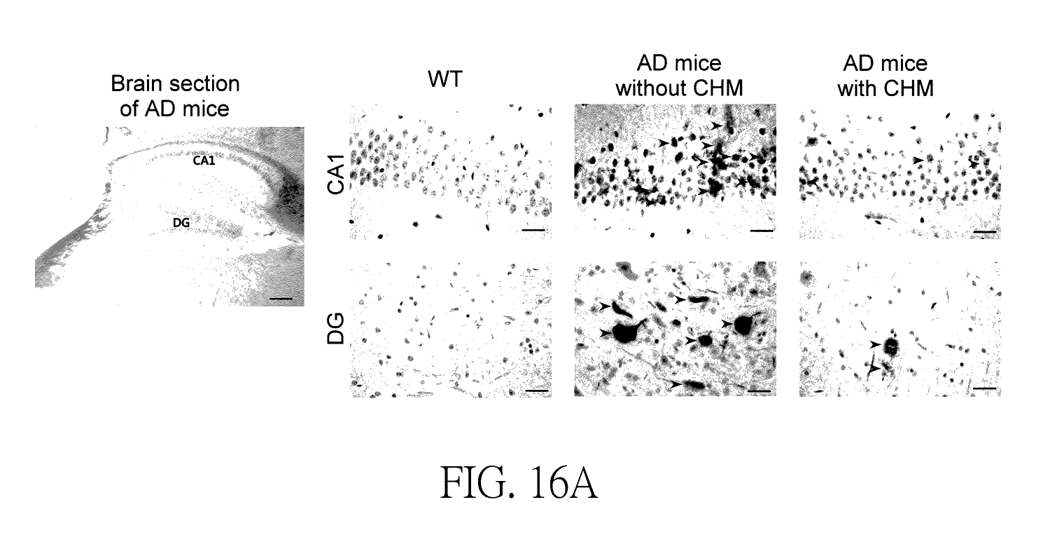

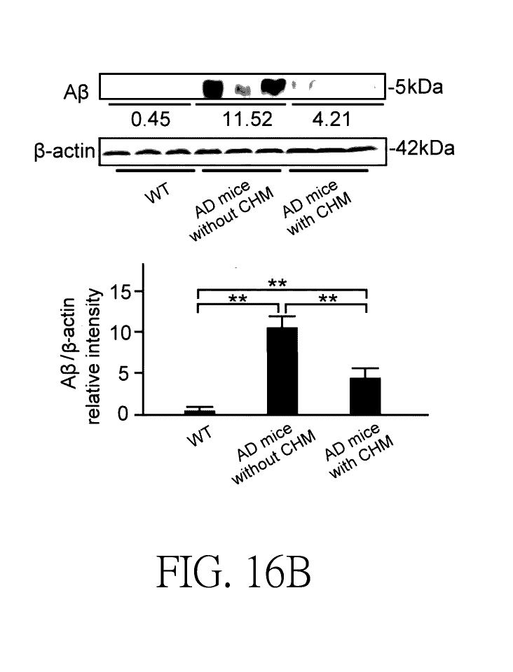

Since Amyloid Beta (AP) deposition is observed in Alzheimer's disease, whether the expression level of AP in the brain can be reduced by the Chinese herbal medicine composition of the present invention is determined. FIGS. 16A-16B show the expression levels of AP in the brains of 9-month AD mice treated with or without the Chinese herbal medicine composition of the present invention and wild type (WT) littermates (6 mice per group). As shown in FIG. 16A, the expression levels of AP in the hippocampal CA1 region (CA1) and the dentate gyrus (DG) of AD mice treated with the Chinese herbal medicine composition of the present invention are reduced (as shown by immunohistochemical staining). As shown in FIG. 16B, the expression levels of AP in the brain tissues of AD mice treated with the Chinese herbal medicine composition of the present invention are significantly reduced (as shown by western blotting) (p<0.01).

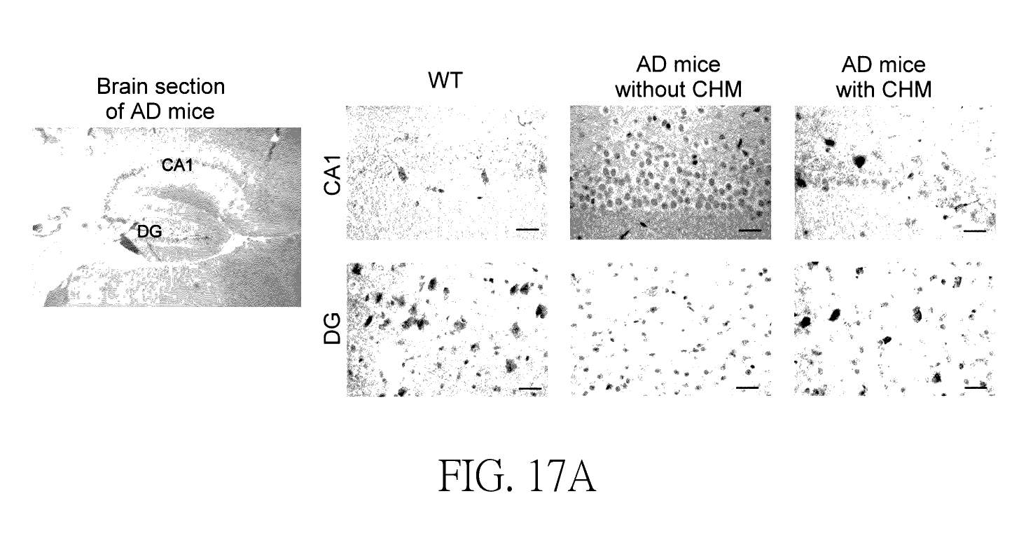

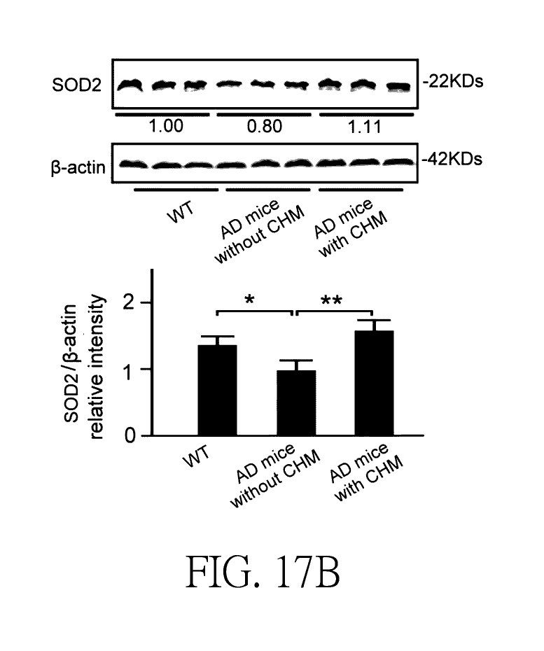

Amyloid Beta (Aβ) deposition will increase oxidative stress in the brain, increasing concentration of free radicals. In the pathway of free radical generation, Superoxide Dismutase 2 (SOD2), which is an important messenger, inhibits the generation of free radicals Reactive Oxygen Species (ROS). As a result, whether the expression level of SOD2 in the brain can be enhanced by the Chinese herbal medicine composition of the present invention is determined. FIGS. 17A-17B show the expression levels of SOD2 in the brains of 9-month AD mice treated with or without the Chinese herbal medicine composition of the present invention and wild type (WT) littermates (6 mice per group). As shown in FIG. 17A, the expression levels of SOD2 in the hippocampal CA1 region (CA1) and the dentate gyrus (DG) of AD mice treated with the Chinese herbal medicine composition of the present invention are enhanced (as shown by immunohistochemical staining). As shown in FIG. 17B, the expression levels of SOD2 in the brain tissues of AD mice treated with the Chinese herbal medicine composition of the present invention are significantly enhanced (as shown by western blotting) (p<0.01-0.05). In other words, the Chinese herbal medicine composition of the present invention can increase the activity of superoxide dismutases and inhibit reactive oxygen species to reduce oxidative stress in brain tissue.

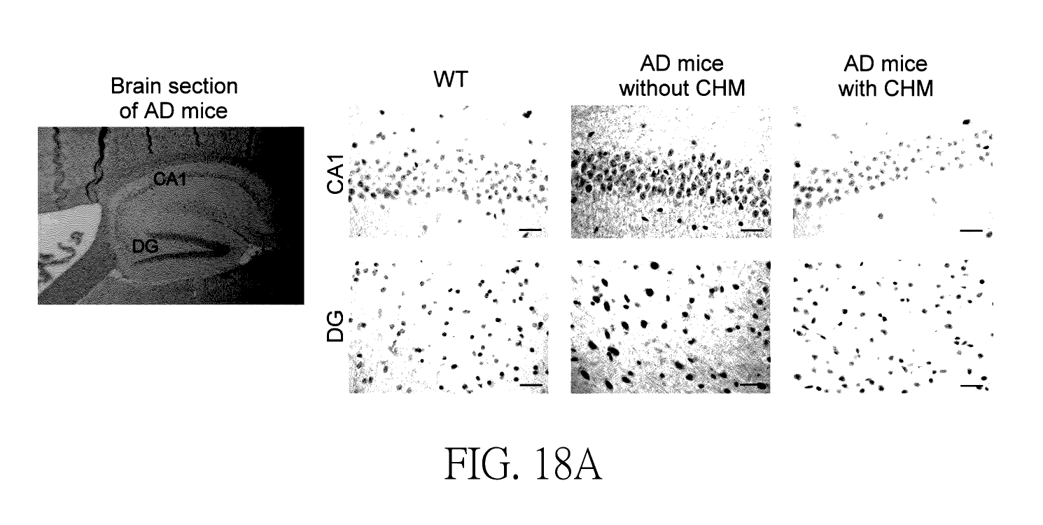

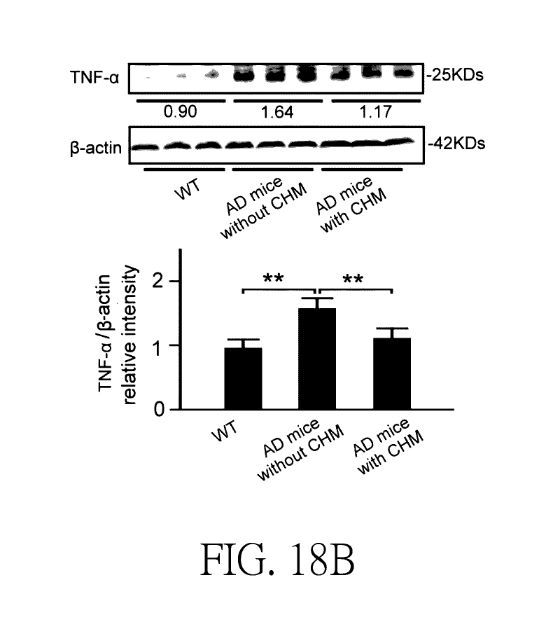

Since inflammation is believed to be involved in the pathogenesis and progression of neurologic diseases and Tumor Necrosis Factor Alpha (TNF-α) is a key marker for inflammation, whether the expression level of TNF-α in the brain can be reduced by the Chinese herbal medicine composition of the present invention is determined. FIGS. 18A-18B show the expression levels of TNF-α in the brains of 9-month AD mice treated with or without the Chinese herbal medicine composition of the present invention and wild type (WT) littermates (6 mice per group). As shown in FIG. 18A, the expression levels of TNF-α in the hippocampal CA1 region (CA1) and the dentate gyrus (DG) of AD mice treated with the Chinese herbal medicine composition of the present invention are reduced (as shown by immunohistochemical staining). As shown in FIG. 18B, the expression levels of TNF-α in the brain tissues of AD mice treated with the Chinese herbal medicine composition of the present invention are significantly reduced (as shown by western blotting) (p<0.01-0.05).

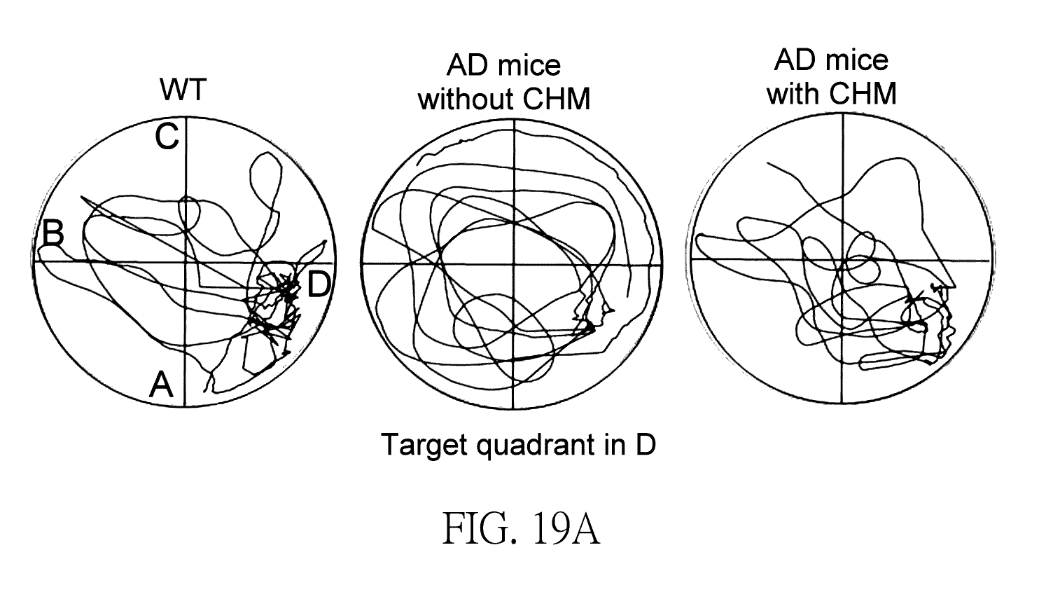

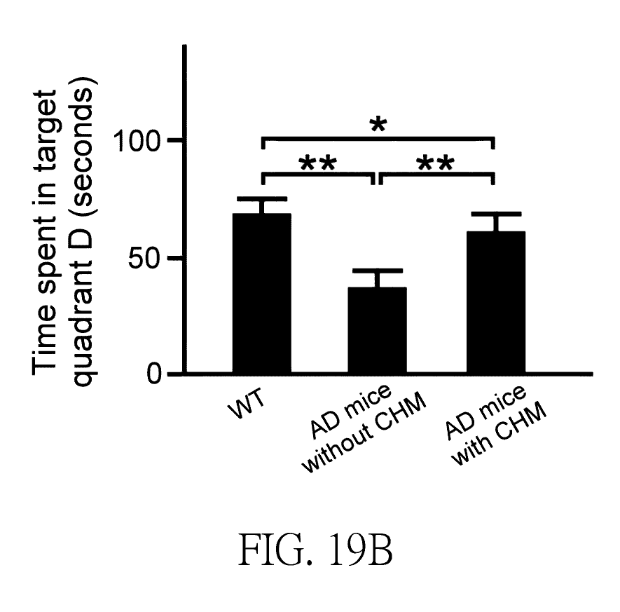

At last, Morris water navigation task, also known as Morris water maze, is conducted to evaluate the memory of AD mice. The Morris water navigation task is used to evaluate if the AD mice can learn or memorize and the ability to learn or memorize. FIG. 19A shows the spatial learning and memorizing abilities of 9-month AD mice treated with or without the Chinese herbal medicine composition of the present invention and wild type (WT) littermates using Morris water maze. As shown in FIG. 19A, the spatial learning and memorizing abilities of AD mice treated with the Chinese herbal medicine composition of the present invention are significantly improved. FIG. 19B shows the statistical analysis of the differences among the times spent at target quadrant D of 9-month AD mice treated with or without the Chinese herbal medicine composition of the present invention and wild type (WT) littermates. As shown in FIG. 19B, the times spent at target quadrant D of AD mice treated with the Chinese herbal medicine composition of the present invention is significantly longer (p<0.01-0.05). As shown, the memory of AD mice treated with the Chinese herbal medicine composition of the present invention is almost the same as that of the wild type (WT) littermates.

### Experiment Example 4

### Parkinson's Disease (PD) Model

In Experiment Example 4, the effect of the Chinese herbal medicine composition of the present invention in the treatment or prevention of Parkinson's disease is explored. Manganese-induced Parkinson's Disease mice (PD mice) are used as test subjects.

Parkinson's disease (PD) is a motor system disorder which is the result of the loss of dopamine-producing brain cells. The PD can be difficult to diagnose accurately. It has been reported that PD patients show markedly decreased dopaminergic levels in their substantia nigra and striatum.

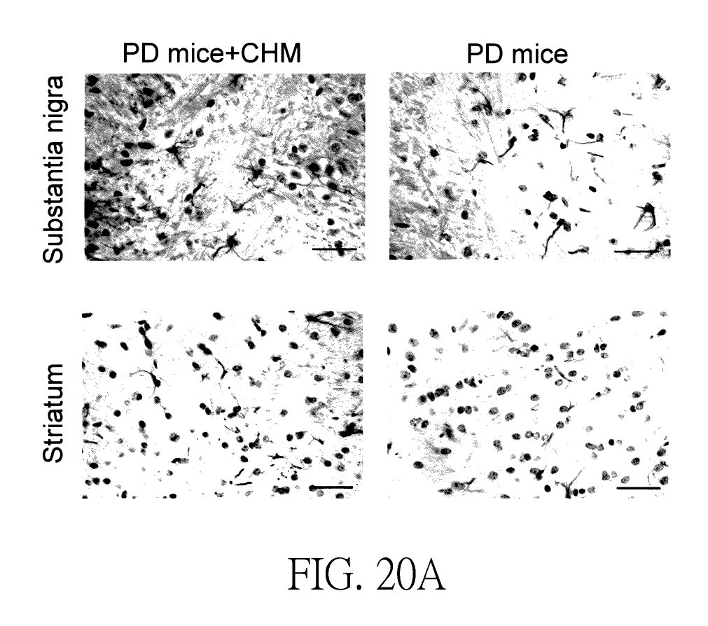

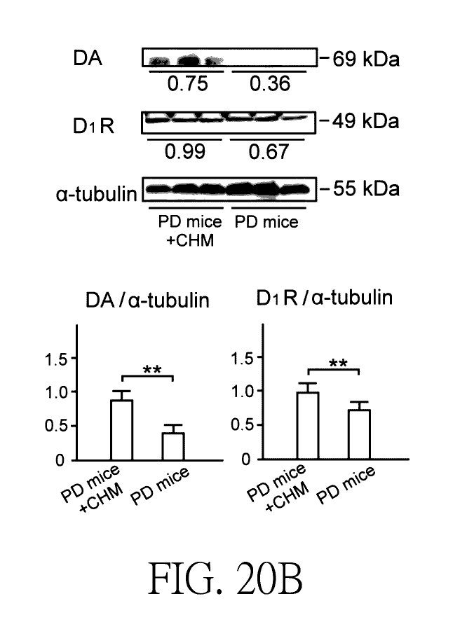

FIGS. 20A-20B show the expression levels of dopaminergic levels (DA) and dopaminergic receptor's (D₁R) levels in the brain tissue of the manganese-induced Parkinson's disease (PD) mice treated with or without the Chinese herbal medicine composition of the present invention. As shown in FIG. 20A, the expression levels of D₁R in the substantia nigra and the striatum of PD mice treated with the Chinese herbal medicine composition of the present invention are increased (as shown by immunohistochemical staining). As shown in FIG. 20B, the expression levels of DA and D₁R in the brain tissues of PD mice treated with the Chinese herbal medicine composition of the present invention are significantly enhanced (as shown by western blotting) (p<0.01).

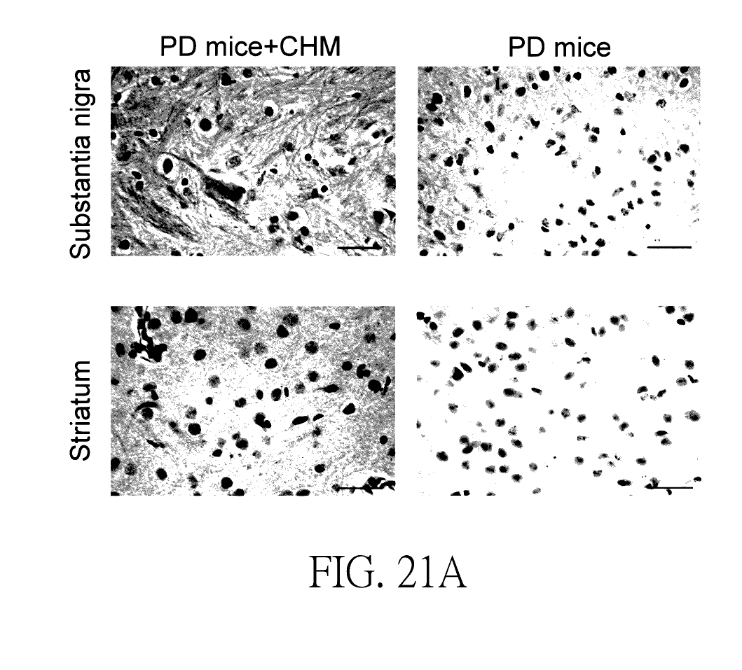

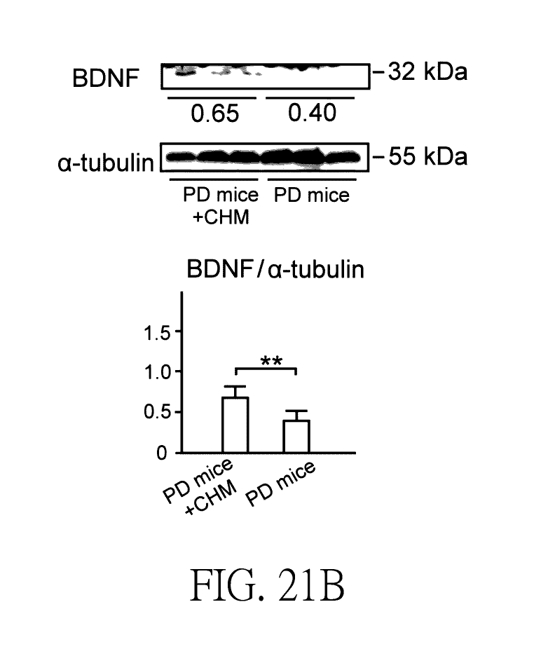

FIGS. 21A-21B show the expression levels of Brain-Derived Neurotrophic Factor (BDNF) in the brains of manganese-induced PD mice As shown in FIG. 21A, the expression levels of BDNF in the substantia nigra and the striatum of PD mice treated with the Chinese herbal medicine composition of the present invention are increased (as shown by immunohistochemical staining). As shown in FIG. 21B, the expression levels of BDNF in the brain tissues of PD mice treated with the Chinese herbal medicine composition of the present invention are significantly enhanced (as shown by western blotting) (p<0.01).

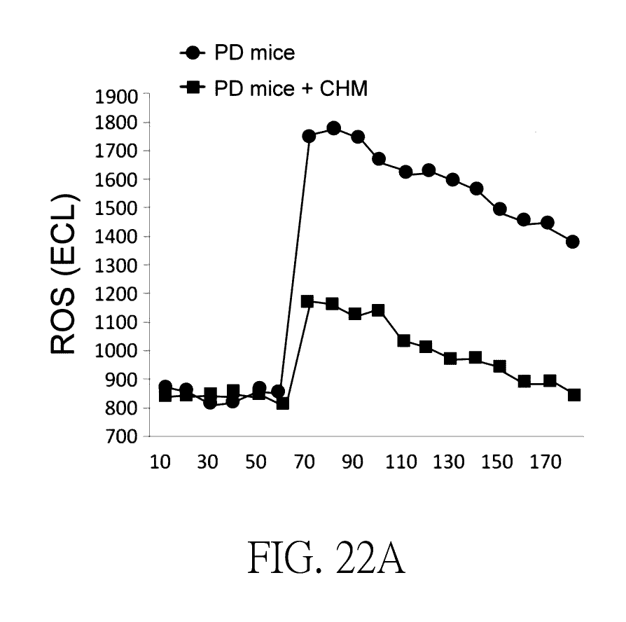

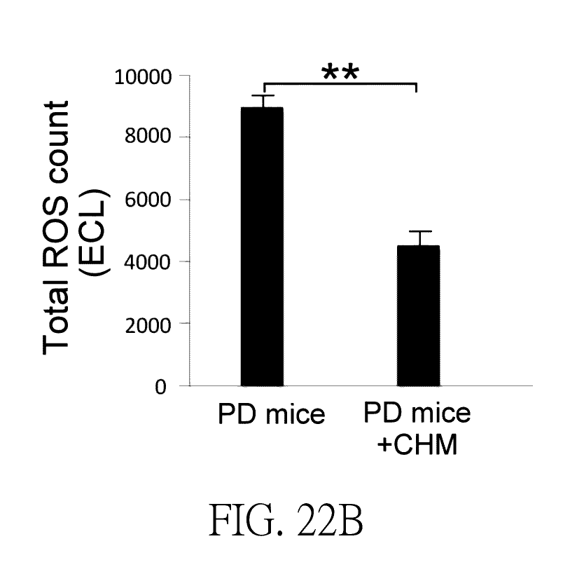

FIGS. 22A-22B show levels of free radicals Reactive Oxygen Species (ROS) in the blood of manganese-induced PD mice treated with or without the Chinese herbal medicine composition of the present invention. As shown in FIG. 22A, the ROS in the blood of manganese-induced PD treated with the Chinese herbal medicine composition of the present invention are decreased with time. As shown in FIG. 22B, the total ROS in the blood of manganese-induced PD treated with the Chinese herbal medicine composition of the present invention are significantly enhanced (as shown by western blotting) (p<0.01).

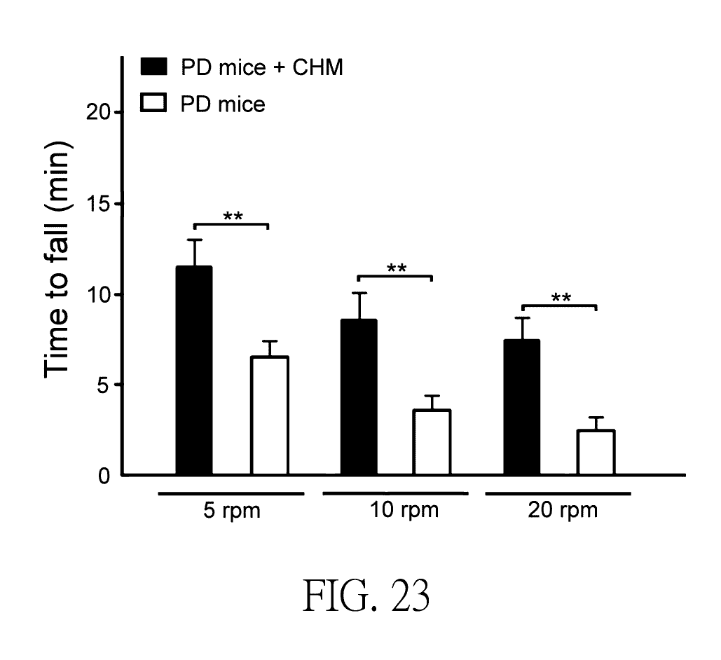

FIG. 23 show the improved motor coordination of the manganese-induced PD mice treated by a Chinese herbal medicine composition of the present invention. By using rotarod testing at different speeds, the latencies to fall of manganese-induced PD treated with the Chinese herbal medicine composition of the present invention are significantly enhanced (as shown by western blotting) (p<0.01).

Accordingly, as shown by the results described above, the Chinese herbal medicine composition of the present invention can increase blood flow and microcirculation of the brain to improve supply of nutrient effectively. The Chinese herbal medicine composition of the present invention can also provide effects such as anti-inflammation and free-radicals elimination to reduce oxidative stress in the brain. The Chinese herbal medicine composition of the present invention can also protect nerve cells and repair damaged neurons. The Chinese herbal medicine composition of the present invention can also reduce stress in endoplasmic reticulum, prevent mitochondria dysfunction, and inhibit apoptosis of neurons. Altogether, the Chinese herbal medicine composition of the present invention can thereby slow down and prevent dementia.

Although the present invention has been explained in relation to its preferred embodiments, it is to be understood that many other possible modifications and variations can be made without departing from the spirit and scope of the invention as hereinafter claimed.

What is claimed is:

1. A method for treating or suppressing degenerative brain diseases, comprising a step of administrating a Chinese herbal medicine composition to a subject in need, wherein the Chinese herbal medicine composition comprises:
a first herbal formula comprising 2-40 parts by weight of Panax ginseng and 0.6-28 parts by weight of Angelica sinensis; and
a second herbal formula comprising 0.6-28 parts by weight of Angelica sinensis, 2-40 parts by weight of Astragalus membranaceus, and 20-96 parts by weight of a mixture of Ligustri fructus, Eclipta prostrata, and Rehmannia glutinosa.

2. The method as claimed in claim 1, wherein the Chinese herbal medicine composition comprises the first herbal formula comprising 20-40 parts by weight of Panax ginseng and 0.8-20 parts by weight of Angelica sinensis and the second herbal formula comprising 1-24 parts by weight of Angelica sinensis, 2-40 parts by weight of Astragalus membranaceus, and 20-96 parts by weight of the mixture of Ligustri fructus, Eclipta prostrata, and Rehmannia glutinosa.

3. The method as claimed in claim 1, wherein a weight ratio of Ligustri fructus, Eclipta prostrata, and Rehmannia glutinosa in the mixture is 0.8-1.2:0.8-1.2:0.8-1.2.

4. The method as claimed in claim 1, wherein the Chinese herbal medicine composition further comprises an excipient selected from a group consisting of starch, corn starch, lactose, microcrystalline cellulose, carboxymethylcellulose, sucrose, mannitol, talcum powder, and magnesium stearate.

5. The method as claimed in claim 1, wherein the Chinese herbal medicine composition further comprising Gastrodiae Rhizoma.

6. The method as claimed in claim 1, wherein the Chinese herbal medicine composition is administered orally.

7. The method as claimed in claim 1, wherein the degenerative brain diseases are at least one of the Huntington's disease, cerebrovascular dementia, Alzheimer's disease, Lewy body dementia, frontotemporal dementia, and Parkinson's disease.

8. The method as claimed in claim 1, wherein the Chinese herbal medicine composition is prepared by the following method, comprising:
(A) providing a first herbal formula and a second herbal formula, wherein the first herbal formula comprises 20-40 parts by weight of Panax ginseng and 0.8-20 parts by weight of Angelica sinensis and the second herbal formula comprises 0.6-28 parts by weight of Angelica sinensis, 2-40 parts by weight of Astragalus membranaceus, and 20-96 parts by weight of a mixture of Ligustri fructus, Eclipta prostrata, and Rehmannia glutinosa;
(B) extracting and filtering the first herbal formula and the second herbal formula to obtain a first extract and a second extract, respectively;
(C) filtering the second extract to obtain a third extract; and
(D) mixing the first extract and the third extract.

9. The method as claimed in claim 8, wherein in step (B), the first herbal formula is extracted using alcohol.

10. The method as claimed in claim 9, wherein the alcohol is ethanol.

11. The method as claimed in claim 9, wherein a concentration of the alcohol is 30-80 vol %.

12. The method as claimed in claim 8, wherein in step (B), the second herbal formula is extracted using water.
